(19) United States
(12) Patent Application Publication
(10) Pub. No.: US 2026/0193612 A1
(43) Pub. Date: Jul. 9, 2026

(54) METHOD OF PREPARING A MASS CULTURE OF MUSCLE PRECURSOR CELLS (MPCS) AND USES THEREOF

(71) Applicant: UNIVERSITÄT ZÜRICH, Zurich (CH)

(72) Inventors: Deana MOHR-HARALAMPIEVA, Kreuzlingen (CH); Jenny Ann PRANGE, Schlieren (CH); Ruud DAS, Utrecht (NL); Marijn DRIESSEN, Amersfoort (NL); Emerentius Gerardus ROOSLOOT, De Meern (NL)

(21) Appl. No.: 19/134,633

(22) PCT Filed: Dec. 1, 2023
(86) PCT No.: PCT/EP2023/083980

§ 371 Date: May 30, 2025

(30) Foreign Application Priority Data
Dec. 1, 2022 (EP) 22210871.4

(51) Int. Cl. C12N 5/077; A61K 35/34

(52) U.S. Cl. CPC C12N 5/0658; A61K 35/34; C12N 2502/115; C12N 2531/00; C12N 2533/54

(57) ABSTRACT

Provided are methods of obtaining a mass culture of muscle derived muscle precursor cells (MPCs) using microcarriers as growth substrate, methods for obtaining a therapeutic efficient amount of those cells, a cell population obtained by said methods as well as compositions comprising the expanded cells and methods for preparing a medicament, for example, for use in the treatment of skeletal muscle dysfunctions.

### FIELD OF THE INVENTION

The invention relates to a method to obtain a mass culture of muscle precursor cells (MPCs) as well as to a cell population comprising MPCs obtained by said method and to a composition comprising said MPCs. Furthermore, the invention is directed to a method for the preparation of a medicament based on the obtained MPCs for use in the treatment of skeletal muscle dysfunctions.

### BACKGROUND OF THE INVENTION

Skeletal muscles damaged by injury or by degenerative diseases such as muscular dystrophy are able to regenerate new muscle fibers, wherein regeneration mainly depends upon myogenic precursor cells. Accordingly, transplantation of muscle precursor cells (MPCs) has been investigated as a treatment for a variety of genetic and acquired muscle disorders. Satellite cells are quiescent adult stem cells and are located under the membrane surrounding the muscle fibers. After trauma or damage, satellite cells get activated as MPCs and participate in tissue regeneration by proliferating and differentiating into myoblasts, which later fuse to form new myofibers. The majority of MPCs are committed to the myogenic lineage and are therefore most suitable for muscle tissue engineering (Eberli et al., Cell Transplant 21 (2012), 2089-98).

In view of their regeneration capacity, MPCs, which are more differentiated than stem cells and further determined towards a muscle lineage, are a promising treatment option in injured, diseased and aged muscle tissue and their potential has been broadly explored. Stress urinary incontinence (SUI), i.e. the involuntary loss of urine due to coughing, laughing, sneezing, exercising and other movements that increase the intra-abdominal pressure on the bladder, is one example of muscle dysfunction, which benefits from cell therapy.

For example, as reviewed by Schmid and colleagues, cell-based therapeutic approaches have been developed to regenerate the sphincter muscle, wherein precursor cells are isolated from living human tissue biopsies, thereafter multiplied in vitro and replanted to repair or replace the injured or diseased tissue (Schmid et al., International Journal of Molecular Sciences 22 (2021), 3981). A particular option is the implantation of autologous muscle precursor cells into the sphincter area to strengthen and restore external urethral sphincter function as also disclosed in WO 2019/215090 A1.

However, one of the limiting steps inter alia in such cell-based therapy for the treatment of muscle dysfunctions is the absence of an efficient method to produce an amount of MPCs sufficiently high for therapeutic applications.

### SUMMARY OF THE INVENTION

The present invention generally relates to a method of obtaining a mass culture of muscle derived precursor cells (MPCs), wherein the cultivation is preferably performed in a 3D cultivation system. In particular, the method of the present invention comprises the cultivation of MPCs in a container comprising culture medium and microcarriers under conditions allowing the MPCs to attach to the microcarriers, wherein the MPCs are preferably seeded at a density between 500-1500 cells/cm² of the growth surface provided by the microcarriers. The method of the present invention further comprises a step of increasing the growth surface area in the culture environment when the cell number from the initial seeding has increased preferably about 8-fold to 25-fold. In one embodiment, the step of increasing the growth area of the method of the present invention is performed when the cell number has increased to about 1.3×10⁴-1.8×10⁴ cells/cm² and/or when more than 80%, preferably more than 90% of the microcarriers are occupied. The method of the present invention further comprises the cultivation of the MPCs until a cell density of preferably up to 5-7.5×10⁴ cells/cm², i.e., of at least or no more than 5-7.5×10⁴ cells/cm², and/or 4-6.5×10⁵ cells/ml has been reached. In a preferred embodiment, the cells are further cultivated after the culture medium has been increased until a cell number of about 1.5-2.75×10⁸ has been obtained. Preferably, the MPCs are obtained from a patient, preferably a human patient as described below.

Accordingly, with the help of the present invention, a culture system was established which allows the generation of a high amount of MPCs. MPCs are anchorage-dependent cells, commonly referred to as adherent cells. These cells need to adhere to a surface in order to remain viable and to proliferate. The method of the present invention has particular advantages in comparison to hitherto applied methods which rely on MPC cultures as monolayers on plates. In particular, high cell yields can be obtained with the growth area provided by the method of the present invention without the use of re-plating steps. Accordingly, the method of the present invention is less laborious and time consuming than conventional monolayer culture systems on plates and the risk of contaminations is also lower when applying the method of the present invention due to the closed system. It is also to be emphasized that since with the current method, a high amount of MPCs is reached much faster than for example in conventional 2D culture systems, advantageously, the obtained population of MPCs is “nearer” to the patient for example in terms of unwanted mutations which are known to be accumulated during the cultivation, i.e. due to the shorter cultivation time the chances are lower that unwanted mutations arise during the cultivation.

A further advantage of the method of the present invention is the low seeding density required for efficient cell expansion. As can be derived for example from WO 2019/215090 A1, a seeding density of 5000 cells/cm² was used to grow the cells, wherein in the method of the present invention, a seeding density of 500-1500 cells/cm² is already sufficient to provide cell growth and results in high yield after the cultivation steps.

As shown in Example 2 and FIGS. 1 and 2, MPCs can efficiently be cultivated and proliferated on microcarriers to obtain cell numbers large enough for downstream applications, e.g. for therapeutic approaches. In order to provide proper growth conditions, the concentration of the cells in the culture medium and their ratio to the growth surface provided by the microcarriers is important. Therefore, in a preferred embodiment, the method of the present invention comprises the seeding of the MPCs at a density between 800-1200 cells/cm². In a further preferred embodiment, the MPCs are seeded at a density between 800-1200 cells/cm² in a culture volume as indicated, infra, preferably in 130 ml culture medium.

As mentioned above, the method of the present invention comprises a step of increasing the growth surface in the culture environment when a certain cell density, cell number and/or fold increase of the cells as defined elsewhere herein has been reached. In a preferred embodiment, the growth surface area and optionally the volume of the culture medium is increased between two and fourfold, preferably threefold. In one embodiment, the starting volume used in the method of the present invention is about 100 to 150 ml and the volume of the culture medium is increased to about 400 ml. In order to obtain higher cell numbers, the step of increasing the growth surface can be repeated one or more times, i.e. the growth surface can again be increased between two and fourfold when a certain cell density, cell number and/or fold increase of the cells as defined elsewhere herein has been reached. Therefore, in one embodiment, the present invention comprises one or more steps of increasing the growth area of the culture environment.

As shown in Examples 1 and 2, a bioreactor system can be used in accordance with the method of the present invention, i.e. MPCs can be cultivated in a bioreactor to obtain a mass culture of the MPCs. Accordingly, in one embodiment the container as used in accordance with the present invention is a closed bioreactor. In a preferred embodiment, the container is a bioreactor bag. In a further embodiment, the container as used in accordance with the method of the present invention is an expandable container. In a preferred embodiment, the container is an expandable bioreactor bag.

As shown by flow cytometry analyses in Example 3, the method of the present invention results in MPCs, i.e. a population comprising MPCs that express myogenic markers. As explained further below, such a population comprises next to MPCs also other cells in different stages during muscle differentiation, for example also cells of early lineages, so that the population is a heterogeneous population. Furthermore, the cultured cells show similar characteristics as the MPCs produced with the method disclosed in WO 2019/215090 A1, in particular in terms of characteristics essential for therapeutic utility, e.g., high expression of Pax7 and α-actinin, and low expression of CD34, which confirms that the cells are therapeutically useful as described, infra.

In accordance with the method of the present invention, microcarriers are used that enable the culture of adherent cells in suspension and provide a huge growth area available for cell growth. As explained above, this is advantageous over the cultivation of MPCs in monolayer culture as conventionally done, since a factor limiting the yield of adherent cell mass culture is the limited growth area in 2D culture systems. Therefore, in one embodiment, the microcarriers are coated microcarriers, preferably collagen-coated microcarriers. In one embodiment, the microcarriers are dissolvable, and in a preferred embodiment, the microcarriers as used in accordance with the present invention are collagen-coated and dissolvable.

In one embodiment, the culture medium as used in accordance with the method of the present invention comprises human platelet lysate (hPL).

In one embodiment, the culture medium as used in accordance with the method of the present invention comprises human platelet lysate (hPL), preferably fibrinogen-depleted hPL and is devoid of heparin and thus, devoid of components that are prone to trigger allergies such as serum or heparin as used in conventional growth medium.

In one embodiment, the culture medium as used in accordance with the method of the present invention comprises human platelet lysate (hPL), and is devoid of heparin and thus, devoid of components that are prone to trigger allergies such as serum or heparin as used in conventional growth medium.

For downstream applications it is important that the cells per se are available without being attached to the microcarriers. Therefore, in one embodiment, the method of the present invention comprises a step of separating the MPCs from the microcarriers at the end of the cultivation. In a preferred embodiment, the separation comprises a complete dissolution of the microcarriers, wherein the dissolution of the microcarriers is preferably performed by enzymatic digestion, preferably by the addition of TrypLE® and pectinase.

The present invention further relates to a cell population comprising MPCs obtainable by the method of the present invention as disclosed herein.

As shown by flow cytometry analyses in Example 3, the cultivation results in MPCs, i.e. a population comprising MPCs that express myogenic markers. Such a population is a heterogenous population and comprises next to MPCs also other cells in different stages during muscle differentiation, for example also cells of early lineages. The presence of cells in different stages during muscle differentiation therefore account for the percentages of cells expressing the specific marker genes shown in Example 3, and FIGS. 4 and 5. As can be seen in FIGS. 3 and 4, the cultivation in the bioreactor resulted in MPCs that express myogenic markers with about 99% of the cells being Pax7, α-Actinin and A2B5 positive and CD34 expression being negative.

In one embodiment, more than 40% of the cells of the population express α-actinin, preferably more than 50%, preferably more than 60%, preferably more than 65%, preferably more than 70%, preferably more than 75%, preferably more than 80%, preferably more than 85%, preferably more than 90%, preferably more than 95%, preferably more than 98%, preferably more than 99% express α-actinin; and/or more than 60% of the cells of the population express Pax7, preferably more than 65%, preferably more than 70%, preferably more than 75%, preferably more than 80%, preferably more than 85%, preferably more than 90%, preferably more than 95%, preferably more than 98%, preferably more than 99% express Pax7; and/or less than 20% of the cells of the population express CD34, preferably less than 15%, preferably less than 10%, preferably less than 8%, preferably less than 7.5%, preferably less than 5%, preferably less than 4%, preferably less than 3%, preferably less than 2%, preferably less than 1.5%, preferably less than 1%, preferably less than 0.5%, preferably less than 0.25% of the cells express CD34.

In one embodiment, more than 40% of the cells of the population express α-actinin, preferably more than 50%, preferably more than 60%, preferably more than 65%, preferably more than 70%, preferably more than 75%, preferably more than 80%, preferably more than 85%, preferably more than 90%, preferably more than 95%, preferably more than 98%, preferably more than 99% express α-actinin; and more than 60% of the cells of the population express Pax7, preferably more than 65%, preferably more than 70%, preferably more than 75%, preferably more than 80%, preferably more than 85%, preferably more than 90%, preferably more than 95%, preferably more than 98%, preferably more than 99% express Pax7; and less than 20% of the cells of the population express CD34, preferably less than 15%, preferably less than 10%, preferably less than 8%, preferably less than 7.5%, preferably less than 5%, preferably less than 4%, preferably less than 3%, preferably less than 2%, preferably less than 1.5%, preferably less than 1%, preferably less than 0.5%, preferably less than 0.25% of the cells express CD34.

In one preferred embodiment, ≥50% of the cells of the population express α-Actinin, ≥60% of the cells of the population express Pax7 and ≤15% of the cells of the population express CD34, i.e., the population comprises ≥50% α-Actinin positive, ≥60% Pax7 positive, and ≤15% CD34 positive (≤15% of the population express CD34) cells.

More preferred, ≥80% of the cells of the population express α-Actinin, ≥80% of the cells of the population express Pax7 and ≤5% of the cells of the population express CD34, i.e., the population comprises ≥80% α-Actinin positive, ≥80% Pax7 positive, and ≤5% CD34 positive cells.

The population of the present invention can be further characterized by its expression of A2B2. Thus, in one embodiment, more than 50% of the cells of the population express A2B5, preferably more than 60%, preferably more than 65%, preferably more than 70%, preferably more than 75%, preferably more than 80%, preferably more than 85%, preferably more than 90%, preferably more than 95%, preferably more than 98%, preferably more than 99%.

Thus, in one embodiment, ≥50% of the cells of the population express α-Actinin, ≥60% of the cells of the population express Pax7, ≥60% of the cells of the population express A2B5, and ≤15% of the cells of the population express CD34, i.e., the population comprises ≥50% α-Actinin positive, ≥60% Pax7 positive, ≥60% A2B5 positive, and ≤15% CD34 positive (≤15% of the population express CD34) cells.

More preferred, ≥80% of the cells of the population express α-Actinin, ≥80% of the cells of the population express Pax7, ≥80% of the cells of the population express A2B5, and ≤5% of the cells of the population express CD34, i.e., the population comprises ≥80% α-Actinin positive, ≥80% Pax7 positive, ≥80% A2B5 positive, and ≤5% CD34 positive cells.

In addition, or alternatively, the population of the present invention is further characterized by comprising cells which express Desmin, preferably wherein between 1% to 99%, preferably between 10% to 90% or between 20% to 80%, preferably up to 75%, preferably up to 70%, preferably between 20% to 70%, or up to 60% of the cells of the population express Desmin.

Thus, in one embodiment, ≥50% of the cells of the population express α-Actinin, ≥60% of the cells of the population express Pax7, ≤15% of the cells of the population express CD34, and wherein the cells express Desmin preferably ≥10%, i.e., the population comprises ≥50% α-Actinin positive, ≥60% Pax7 positive, and ≤15% CD34 positive (≤15% of the population express CD34) cells, and wherein the population comprises cells that express Desmin, preferably ≥10%.

More preferred, ≥80% of the cells of the population express α-Actinin, ≥80% of the cells of the population express Pax7, and ≤5% of the cells of the population express CD34, and wherein the cells express Desmin, preferably ≥10% Desmin, i.e., the population comprises ≥80% α-Actinin positive, ≥80% Pax7 positive, and ≤5% CD34 positive cells, and wherein the population comprises cells that express Desmin, preferably ≥10%.

In one embodiment, ≥50% of the cells of the population express α-Actinin, ≥60% of the cells of the population express Pax7, ≥60% of the cells of the population express A2B5, ≤15% of the cells of the population express CD34, and wherein the cells express Desmin preferably ≥10%, i.e., the population comprises ≥50% α-Actinin positive, ≥60% Pax7 positive, ≥60% A2B5 positive, and ≤15% CD34 positive (≤15% of the population express CD34) cells, and wherein the population comprises cells that express Desmin, preferably ≥10%.

More preferred, ≥80% of the cells of the population express α-Actinin, ≥80% of the cells of the population express Pax7, ≥80% of the cells of the population express A2B5, and ≤5% of the cells of the population express CD34, and wherein the cells express Desmin, preferably ≥10% Desmin, i.e., the population comprises ≥80% α-Actinin positive, ≥80% Pax7 positive, ≥80% A2B5 positive, and ≤5% CD34 positive cells, and wherein the population comprises cells that express Desmin, preferably ≥10%.

Moreover, as can be seen in FIG. 5, beside displaying the typical myogenic markers α-Actinin and A2B5 (99.9% and 99.7% respectively) and CD34 being negative (0.1%), cells cultivated in the bioreactor were also positive for expression of Myf5, myHC and MyoD (67.6%, 8.7% and 19.6% respectively), and show very low expression of CD56 (3.3%).

Accordingly, the population of the present invention can be further characterized by comprising more than ≤15% CD56 positive cells, ≥50% Myf5 positive cells, ≤30% MyHC positive cells, and/or 10-40% MyoD positive cells, preferably the cell population comprises ≤10% CD56 positive cells, ≥60% Myf5 positive cells, ≤20% MyHC positive cells and/or 10-30% MyoD positive cells, most preferably the cell population comprises ≤5% CD56 positive cells, ≥60-90% Myf5 positive cells, ≤15% MyHC, and/or 15-25% MyoD positive cells.

In more detail:

The population of the present invention can be further characterized by its expression of MyHC. In particular, in one embodiment, between 0% and 29% of the cells of the population express MyHC, and thus, in one embodiment, ≤29% of the cells of the population express MyHC, preferably ≤25%, preferably ≤20%, preferably ≤15%, more preferably ≤10% of the cells express MyHC.

Thus, in one embodiment, ≥50% of the cells of the population express α-Actinin, ≥60% of the cells of the population express Pax7, ≤15% of the cells of the population express CD34, and ≤29% of the cells of the population express MyHC, i.e., the population comprises ≥50% α-Actinin positive, ≥60% Pax7 positive, ≤15% CD34 positive, and ≤29% MyHC positive cells.

More preferred, ≥80% of the cells of the population express α-Actinin, ≥80% of the cells of the population express Pax7, ≤5% of the cells of the population express CD34, and ≤15% of the cells express MyHC, i.e., the population comprises ≥80% α-Actinin positive, ≥80% Pax7 positive, ≤5% CD34 positive, and ≤15% MyHC positive cells.

In one embodiment, ≥50% of the cells of the population express α-Actinin, ≥60% of the cells of the population express Pax7, ≥60% of the cells of the population express A2B5, ≤15% of the cells of the population express CD34, and ≤29% of the cells of the population express MyHC, i.e., the population comprises ≥50% α-Actinin positive, ≥60% Pax7 positive, ≥60% A2B5 positive, ≤15% CD34 positive, and ≤29% MyHC positive cells.

More preferred, ≥80% of the cells of the population express α-Actinin, ≥80% of the cells of the population express Pax7, ≥80% of the cells of the population express A2B5, ≤5% of the cells of the population express CD34, and ≤15% of the cells express MyHC, i.e., the population comprises ≥80% α-Actinin positive, ≥80% Pax7 positive, ≥80% A2B5 positive, ≤5% CD34 positive, and ≤15% MyHC positive cells.

Preferably, the cells of the population of the present invention further express Desmin as indicated above.

The population of the present invention can be further characterized by its expression of MyoD. In particular, in one embodiment, between 10% and 40%, preferably between 10 and 30%, preferably between 15% and 30%, more preferably between 15% to 25% of the cells of the population express MyoD.

Thus, in one embodiment, ≥50% of the cells of the population express α-Actinin, ≥60% of the cells of the population express Pax7, ≤15% of the cells of the population express CD34, and between 10% and 40% of the cells of the population express MyoD, i.e., the population comprises ≥50% α-Actinin positive, ≥60% Pax7 positive, ≤15% CD34 positive, and between 10% and 40% MyoD positive cells.

More preferred, ≥80% of the cells of the population express α-Actinin, ≥80% of the cells of the population express Pax7, ≤5% of the cells of the population express CD34, and between 10% and 30% of the cells of the population express MyoD, i.e., the population comprises ≥80% α-Actinin positive, ≥80% Pax7 positive, ≤5% CD34 positive, and between 10% and 30% MyoD positive cells.

In one embodiment, ≥50% of the cells of the population express α-Actinin, ≥60% of the cells of the population express Pax7, ≥60% of the cells of the population express A2B5, ≤15% of the cells of the population express CD34, and between 10% and 40% of the cells of the population express MyoD, i.e., the population comprises ≥50% α-Actinin positive, ≥60% Pax7 positive, ≥60% A2B5 positive, ≤15% CD34 positive, and between 10% and 34% MyoD positive cells.

More preferred, ≥80% of the cells of the population express α-Actinin, ≥80% of the cells of the population express Pax7, ≥80% of the cells of the population express A2B5, ≤5% of the cells of the population express CD34, between 10% and 30% of the cells of the population express MyoD, i.e., the population comprises ≥80% α-Actinin positive, ≥80% Pax7 positive, ≥80% A2B5 positive, ≤5% CD34 positive, and between 10% and 30% MyoD positive cells.

In one embodiment, ≥50% of the cells of the population express α-Actinin, ≥60% of the cells of the population express Pax7, ≤15% of the cells of the population express CD34, ≤29% of the cells of the population express MyHC, and between 10% and 40% of the cells of the population express MyoD, i.e., the population comprises ≥50% α-Actinin positive, ≥60% Pax7 positive, ≤15% CD34 positive, ≤29% MyHC positive cells, between 10% and 40% MyoD positive cells.

More preferred, ≥80% of the cells of the population express α-Actinin, ≥80% of the cells of the population express Pax7, ≤5% of the cells of the population express CD34, ≤15% of the cells express MyHC, and between 10% and 30% of the cells of the population express MyoD, i.e., the population comprises ≥80% α-Actinin positive, ≥80% Pax7 positive, ≤5% CD34 positive, ≤15% MyHC positive cells, between 10% and 30% MyoD positive cells.

In one embodiment, ≥50% of the cells of the population express α-Actinin, ≥60% of the cells of the population express Pax7, ≥60% of the cells of the population express A2B5, ≤15% of the cells of the population express CD34, ≤29% of the cells of the population express MyHC, and between 10% and 40% of the cells of the population express MyoD, i.e., the population comprises ≥50% α-Actinin positive, ≥60% Pax7 positive, ≥60% A2B5 positive, ≤15% CD34 positive, ≤29% MyHC positive cells, between 10% and 40% MyoD positive cells.

More preferred, ≥80% of the cells of the population express α-Actinin, ≥80% of the cells of the population express Pax7, ≥80% of the cells of the population express A2B5, ≤5% of the cells of the population express CD34, ≤15% of the cells express MyHC, and between 10% and 30% of the cells of the population express MyoD, i.e., the population comprises ≥80% α-Actinin positive, ≥80% Pax7 positive, ≥80% A2B5 positive, ≤5% CD34 positive, ≤15% MyHC positive cells, between 10% and 30% MyoD positive cells.

Preferably, the cells of the population of the present invention further express Desmin as indicated above.

The population of the present invention can be further characterized by its expression of CD56. In particular, in one embodiment, between 0% and 15% of the cells of the population express CD56, preferably between 5% and 15%, and thus, in one embodiment, ≤15% of the cells of the population express CD56, preferably ≤10%, more preferably ≤5% of the cells express CD56.

Thus, in one embodiment, ≥50%, preferably ≥80% of the cells of the population express α-Actinin, ≥60%, preferably ≥80% of the cells of the population express Pax7, ≤15%, preferably ≤5% of the cells of the population express CD34, and ≤15% of the cells of the population express CD56.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≥60%, preferably ≥80% of the cells of the population express A2B5, and ≤15% of the cells of the population express CD56.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyOD, and ≤15% of the cells of the population express CD56.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyOD, and ≤15% of the cells of the population express CD56.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≤29%, preferably ≤15% of the cells of the population express MyHC, and ≤15% of the cells of the population express CD56.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≥60%, preferably ≥80% of the cells of the population express A2B5, ≤29%, preferably ≤15% of the cells of the population express MyHC, and ≤15% of the cells of the population express CD56.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyoD, ≤29%, preferably ≤15% of the cells of the population express MyHC, and ≤15% of the cells of the population express CD56.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyoD, ≤29%, preferably ≤15% of the cells of the population express MyHC, and ≤15% of the cells of the population express CD56.

Preferably, the cells of the population of the present invention further express Desmin as indicated above.

The population of the present invention can be further characterized by its expression of Myf5.

In particular, in one embodiment, ≥50% of the cells of the population express Myf5, preferably ≥60% of the cells of the population express Myf5, preferably between 60% and 89%, more preferably between 65% and 89% of the cells of the population express Myf5.

Thus, in one embodiment, ≥50%, preferably ≥80% of the cells of the population express α-Actinin, ≥60%, preferably ≥80% of the cells of the population express Pax7, ≤15%, preferably ≤5% of the cells of the population express CD34, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≥60%, preferably ≥80% of the cells of the population express A2B5, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyOD, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≤29%, preferably ≤15% of the cells of the population express MyHC, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, ≤15% of the cells of the population express CD56, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyoD, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, ≤29%, preferably ≤15% of the cells of the population express MyHC, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, ≤15% of the cells of the population express CD56, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyoD, ≤29%, preferably ≤15% of the cells of the population express MyHC, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyoD, ≤29%, preferably ≤15% of the cells of the population express MyHC, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyoD, ≤15% of the cells of the population express CD56, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyoD, ≤15% of the cells of the population express CD56, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, ≤29%, preferably ≤15% of the cells of the population express MyHC, ≤15% of the cells of the population express CD56, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≤15%, preferably ≤5% of the cells of the population express CD34, between 10% and 40%, preferably between 10% and 30% of the cells of the population express MyoD, ≤15% of the cells of the population express CD56, ≤29%, preferably ≤15% of the cells of the population express MyHC, and ≤50% of the cells of the population express Myf5, preferably between 60% and 89% of the cells of the population express Myf5.

In one embodiment, ≥50%, preferably ≥80% of the cells of the population express α-Actinin, ≥60%, preferably ≥80% of the cells of the population express Pax7, ≥60%, preferably ≥80% of the cells of the population express A2B5, ≤15%, preferably ≤5% of the cells of the population express CD34, between 10% and 40%, preferably between 10% and 30% of the cells of the population express MyoD, ≤15% of the cells of the population express CD56, ≤ 29%, preferably ≤15% of the cells of the population express MyHC, and ≤50% of the cells of the population express Myf5, preferably between 60% and 89% of the cells of the population express Myf5.

Preferably, the cells of the population of the present invention further express Desmin as indicated above.

As mentioned above, the population of the present invention can be characterized by comprising ≥50%, preferably ≥80% cells that express α-Actinin, ≥60%, preferably ≥80% cells that express Pax7, ≤15%, preferably ≤5% cells that express CD34.

In one embodiment, the population can be further characterized by comprising ≤29%, preferably ≤15% cells that express MyHC.

The low expression of MyHC, which is a contractile protein marker and expressed in more differentiated cell populations, shows that the majority of the cells is in an early stage, i.e., not differentiated.

In addition, or alternatively, the population can be further characterized by comprising cells that express MyoD, preferably the population comprises between 10% and 40%, preferably between 10% and 30% cells that express MyoD. The population can be further characterized by comprising between 60% and 89% of cells that express Myf5.

Quiescent satellite cells are characterized by the expression of Pax7 and the absence of MyoD expression, wherein activated satellite cells express MyoD and/or Myf5. Thus, as can be derived from the marker expression data, for example from the presence of Pax7 and MyoD/Myf5 positive cells, the population of the present invention comprises a mix of activated satellite cells, which are still able to dedifferentiate back to dormant ones to fill up the pool for a potential future injury of the muscle.

In addition, or alternatively, the population is further characterized by comprising ≤15% cells that express CD56. The low expression of CD56, which is a pure myoblast marker, shows that the population is in an early differentiation state.

The population can be further characterized by comprising ≥60%, preferably ≥80% cells that express A2B5. Optionally, but preferably, the population further comprises cells that express Desmin.

In a preferred embodiment, the cell population of the present invention comprises the MPCs in a therapeutically effective amount, which is preferably at least 1×10⁶ MPCs, preferably at least 1×10⁷ MPCs. In a preferred embodiment, the population comprises at least 1-3×10⁸ MPCs.

The present invention also encompasses a method of preparing a medicament comprising the steps of the method of obtaining a mass culture of MPCs of the present invention as disclosed herein, and optionally adding a biomaterial solution, preferably a hydrogel solution, more preferably a collagen solution, most preferably in a final concentration of 1-4 mg/mL, preferably 2 mg/mL, to the harvested MPCs. In one embodiment, the method further comprises a step of filling the MPCs into a pharmaceutical container, which is preferably a syringe or vial.

Furthermore, the present invention relates to a composition comprising the MPCs obtainable by the method of obtaining a mass culture of MPCs of the present invention as disclosed herein. In accordance with the present invention, the composition is used as a medicament. In a preferred embodiment, the present invention relates to a composition for use in the treatment of a muscle dysfunction, preferably wherein the muscle dysfunction is a skeletal muscle dysfunction, more preferably wherein the skeletal muscle dysfunction is a defect of a sphincter muscle, preferably the external urethral sphincter muscle. Optionally the composition of the present invention further comprises biomaterial solution, preferably a hydrogel solution, more preferably a collagen solution, which is preferably mixed with the MPCs, most preferably in the above-indicated concentration.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Further embodiments of the present invention will be apparent from the description and Examples that follow.

For the avoidance of any doubt it is emphasized that the expressions “in some embodiments”, “in a certain embodiments”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

The term “between” includes the endpoints.

### BRIEF DESCRIPTION OF THE DRAWINGS

FIG. 1: Quantification of cultivated MPCs (passage 3). The growth curve of the cell counts presented in Table 1 is given in FIG. 1, i.e. of the cell counts adjusted to account for the loss of biomass due to sampling.

FIG. 2: Visual inspection of cultivated MPCs at 40- and 100-fold magnification.

FIG. 3: Flow cytometry analysis of MPCs cultivated according to the present invention in comparison to the cultivation in monolayer flasks with cells being cultured in the presence of 5% (A) and 10% (B) hPL. The expression of the markers Pax7, α-Actinin, and CD34 was analyzed.

FIG. 4: Flow cytometry analysis of MPCs cultivated according to the present invention in comparison to the cultivation in monolayer flasks. The expression of the markers Pax7, α-Actinin, CD34, and A2B5 was analyzed.

FIG. 5: Flow cytometry analysis of MPCs cultivated according to the present invention in comparison to the cultivation in monolayer flasks. The expression of the markers α-Actinin, A2B5, CD34, CD56, Myf5, MyHC, and MyoD was analyzed.

### DETAILED DESCRIPTION OF THE INVENTION

The present invention relates to a method of obtaining muscle precursor cells (MPCs), which includes cultivating and expanding the MPCs, preferably in a 3D cultivation system. More specifically, the method of the present invention relates to a method for obtaining a population comprising MPCs in a therapeutically effective amount as defined, infra. In particular, the present invention relates to a method of obtaining MPCs which includes the growth of MPCs on microcarriers in a suspension culture. Microcarriers are particles that have a high surface to volume ratio. The surface provided by microcarriers serves as culture support for adherent cells, for which reason efficient expansion of adherent cells in a small culture volume is possible. In accordance with the present invention, MPCs are cultured in growth medium under conditions allowing the MPCs to attach to the microcarriers and to expand thereon. Once a certain density of the cells attached to the microcarriers is reached, the growth surface provided by the microcarriers is increased. The cells are cultured until the desired cell number is reached.

Every type of cells requires distinct cultivation conditions and a process established for one cell type usually cannot be used for another cell type but a new process has to be established. Furthermore, culture conditions for obtaining a mass culture can hardly be predicted. For example, spinner flask cultivations have been performed, wherein MPCs were grown in culture medium comprising microcarriers. For microcarrier based expansion, cells need to migrate from one microcarrier to another and in this process, it is necessary to maintain a homogenous distribution of cells on microcarriers in order to reach high cell yield. In this context, it was found out that a seeding density of about 5000 cells/cm² was optimal to reach high cell yields; see poster presentation by Burer et al., “Optimization of Microcarrier-based Culture of Muscle Precursor Cells”, Scinus Cell Expansion. Accordingly, it is surprising that a low seeding density of only 500-1500 cells/cm² as used in accordance with the present invention is already sufficient to provide sufficient cell growth and results in high yield.

The cultivation system used in accordance with the present invention, e.g. the cultivation system as described in Example 1, has been specifically designed and adapted to grow and expand MPCs. In particular, it has been figured out that the seeding density, i.e. the initial concentration of the cells in the culture medium and their ratio to the growth surface provided by the microcarriers, is important for a successful cultivation. Accordingly, seeding is performed at a density between 500-1500 cells/cm², preferably at a density between 800-1200 cells/cm², and most preferably about 900 cells/cm². Thus, in a most preferred embodiment, the cell concentration for inoculation is about 7500 cells/mL with a microcarrier concentration of 1.7 g/L. This seeding density is especially suitable when about 10⁵ to 10⁶ cells, preferably 10⁶ cells are used for inoculation of 130 mL culture medium. The seeding density has been specifically adapted in view of the low donor cell number to work well with the intended MPC protocol. Another important factor is the expansion, wherein the cell density that allows successful expansion of cells is different for each cell type. Accordingly, the range of cell densities that allows successful expansion of MPCs on microcarriers has been established and when the cell number has increased about 8-fold to 25-fold, preferably when a density between 1.3×10⁴-1.8×10⁴ cells/cm² (i.e. 1.1×10⁵-1.5×10⁵ cells/mL) is reached and/or more than 80%, preferably 90% of the microcarriers are occupied, the surface area is increased, preferably about 3-fold. This will be after approximately three days. The end of the cultivation is a further critical parameter. In particular, the cultivation is ended when a cell density is reached, which provides sufficient cells for further applications, like the below mentioned therapeutic application. Preferably, a cell density of up to 5-7.5×10⁴ cells/cm² (4-6.5×10⁵ cells/ml), preferably of up to 6.7×10⁴ cells/cm² (5.7×10⁵ cells/mL) has been reached. This will be approximately after six days of cultivation. More cells can be obtained by further expansion steps, i.e. by further increasing the growth area, preferably about 3-fold, when more than 80%, preferably 90% of the microcarriers are occupied or a cell density between 1.3×10⁴-1.8×10⁴ cells/cm² (i.e. 1.1×10⁵-1.5×10⁵ cells/mL) is reached. The person skilled in the art will appreciate that such expansion steps can be further repeated every time such occupancy and/or cell density are reached.

### Muscle Precursor Cells

The term “muscle precursor cells” or “MPCs” or just “cells” (if not indicated otherwise) as used herein refers to the pool of all muscle derived precursor cells which express muscle-specific markers and are able to give rise to new myofibers, such as defined for example by Eberli et al., Methods 47 (2009), 98-103. MPCs are also referred to as proliferating satellite cells. The term “population of MPCs” or “population comprising MPCs” or “MPCs” and the like means that MPCs represent the main cell type of the population. However, a population of MPCs may comprise other cell types beside MPCs, i.e. a population of MPCs comprises preferably at least 60% MPCs, at least 65% MPCs, at least 70% MCs, at least 75% MPCs, at least 80% MPCs, at least 85% MPCs, at least 90% MPCs, at least 95% MPCs, at least 98% MPCs, at least 99% MPCs or about 100% MPCs. For example, a population of MPCs might comprise myofibroblasts besides MPCs and is still considered as population of MPCs according to this definition. In addition, a cell population is considered to be a population of MPCs when myogenic markers can be detected as described herein, i.e. the presence of for example α-Actinin, Pax7, A2B5 and absence of CD34; see the section “Cell population and therapeutic aspects” herein for details regarding marker expression.

The MPCs to be cultivated in accordance with the method of the present invention and to be obtained with the method of the present invention, respectively can be derived from the muscle tissue of any species, preferably of a mammalian, more preferably of a domestic animal, i.e. a pet or a livestock (farm animal), or a human. Pets include but are not limited to dogs, cats, rabbits, guinea pigs, hamsters, and horses. Livestock include but are not limited to cattle, cows, pigs, sheep, goats, donkeys, camels, buffaloes, and elephants. Most preferably, the method of the present invention is used to obtain human MPCs (hMPCs). Accordingly, the present invention relates to a method of obtaining a mass culture of MPCs, wherein the MPCs are preferably mammalian MPCs, more preferably domestic animal MPCs, for example pet MPCs or livestock MPCs as defined before, and most preferably human MPCs (hMPCs). The MPCs are preferably skeletal muscle derived and are preferably taken from a healthy muscle preferably from a tissue selected from the group consisting of: musculus soleus, rectus abdominis, quadriceps femoris, vastus lateralis, and vastus intermedius. In case the MPCs obtained by the method of the present invention are intended to be used in the treatment of a skeletal muscle dysfunction as outlined in the section “Cell population and therapeutic aspects”, infra, the person skilled in the art can easily conceive that depending on the target muscle, i.e. the damaged muscle to be treated, the biopsy is taken from a healthy muscle with similar architecture. For example, slow twitch muscle fibers are similar to sphincter muscles and the soleus muscle contains predominantly such slow twitch fibers. Accordingly, in one embodiment of the present invention, the MPCs are obtained from slow twitch muscle fibers, and preferably from the musculus soleus (of the left or right leg) which is similar in composition to the sphincter muscle and is easily accessible. As an alternative, the vastus lateralis muscle can be used. Accordingly, the MPCs as obtained by the method of the present invention are preferably slow twitch muscle fibers derived MPCs, preferably musculus soleus derived MPCs, rectus abdominis derived MPCs, quadriceps femoris derived MPCs, vastus lateralis derived MPCs, or vastus intermedius derived MPCs, and most preferably musculus soleus derived MPCs, or vastus lateralis derived MPC, in particular musculus soleus derived MPCs. Depending on the muscle to be target with the MPC of the present invention, the biopsy can be taken from a fast twitch muscle, for example, if the target muscle is a fast twitch muscle.

MPCs to be cultivated in accordance with the method of the present invention to obtain a corresponding mass culture can be obtained by different ways. A preferred method of isolating MPCs is described in WO 2019/115790 A1 and those cells can be used as inoculum in the method of the present invention and thus, the method of the present invention can be used to obtain a mass culture of those cells. In a preferred embodiment, the MPCs cultivated in accordance with the method of the present invention are isolated as described in WO 2019/215090 A1, in particular in Example 1, which content is incorporated herein by reference.

Accordingly, in a preferred embodiment, in order to isolate the MPCs, a muscle biopsy is taken from a muscle tissue, preferably from a skeletal muscle and more preferably taken from a predominantly slow twitch or fast twitch muscle tissue, preferably from a slow twitch muscle, and most preferably selected from the non-limiting group consisting of: musculus soleus, rectus abdominis, quadriceps femoris, vastus lateralis, vastus intermedius. In a further preferred embodiment, a biopsy is taken from musculus soleus or vastus lateralis and most preferably from musculus soleus.

In one particular embodiment, fat-, and/or tendon-, and/or connective tissue is removed from the human tissue sample and the biopsy is cut into small pieces, preferably by using a scissor, resulting in a viscous mix and digested, preferably by a mixture containing one or more enzymes to disaggregate the tissue, preferably collagenase and dispase. Preferably, a mixture of about 0.05% to 2%, more preferably of about 0.2% collagenase type I (w/v) and about 0.1% to 2%, more preferably of about 0.4%-1.6% dispase (w/v) is used. The enzymatic reaction is preferably performed at 36-38° C. for 15 to 75 min, preferably for 45 to 75 min. The digestion is terminated once the desired degree of digestion is reached, preferably by the addition of cell culture medium, i.e. growth medium as defined herein; see section “culture medium”, infra. In one embodiment, the step of cutting the biopsy is preceded by a step of disinfecting the biopsy using a disinfectant and washed with PBS.

After addition of the growth medium, the digest is mixed, preferably by pipetting and centrifuged. After centrifugation, the pellet is re-suspended, preferably by pipetting up and down, in growth medium. In one embodiment, the growth medium comprises 1% penicillin/streptomycin, preferably 1% (supplemented only for this passage 0 step). In one embodiment, the growth medium is free of penicillin/streptomycin. In an alternative embodiment, the growth medium comprises one or more antibiotic agents other than penicillin or streptomycin, for example gentamycin. The cell suspension is filtered through a strainer, preferably with a pore size of 100 μm. Afterwards the cells are seeded on coated dishes, in particular, the cell suspension is transferred into culture plates such as 35 mm-dishes (6-well), coated with an extracellular matrix protein, such as collagen, fibronectin or laminin, preferably collagen, most preferably collagen type I.

In one embodiment of the present invention, plates, on which cells obtained from a muscle biopsy are to be cultured, are coated with a collagen solution, preferably a collagen type I solution, of about 0.03-1.5 mg/ml, preferably of about 0.05-1 mg/ml, more preferably of 0.05 mg/ml. The collagen solution is transferred to the culture plate so that the bottom of the well is covered with the solution. Afterwards, the collagen solution is removed and the coated plates are washed with PBS for 3 times.

As used herein the term “collagen-coated plate(s)” or “plate(s)” is not limited to culture plates, but also includes culture dishes, in general, which are suitable for the cultivation of cells as a monolayer, such as cell culture flasks. Alternatively, in further embodiments of the present invention, the cells to be expanded from the biopsy are cultured as multilayer, for example in multilayer flasks or in any other 2D cultivation system, or in any 3D cultivation system, for example on microcarriers in spinner flasks.

After seeding the cells on plates coated with an extracellular matrix protein, such as fibronectin or collagen as outlined above, the cells are incubated under appropriate culture conditions, preferably at 36-38° C. and 5% CO₂ for about 20 to 28 h, preferably for 24 h. Afterwards, the supernatant containing non-adhered cells, mostly MPCs is re-plated into dishes coated with an extracellular matrix protein, such as collagen or fibronectin, preferably collagen, most preferably collagen type I, in order to reduce the number of myofibroblasts. The plates are coated as outlined above. The MPCs are allowed to settle in the coated dish, thereby yielding a population comprising MPCs, preferably human MPCs. These cells are regarded as passage 0 (P0) MPCs.

The growth medium is preferably exchanged for the first time after 2 to 4 days and then every 2 to 4 days. In case enough cells for seeding into the mass culture system are already obtained in P0, i.e. about 10⁵ to 10⁶ cells, preferably 10⁶ cells, the MPCs, i.e., the population comprising the MPCs, are directly transferred into the container for mass cultivation. Accordingly, in one embodiment, the MPCs to be cultured in accordance with the method of the present invention are passage 0 (P0) cells, which are preferably obtained as described hereinbefore.

Otherwise, the MPCs are split. For splitting, the MPCs are washed with PBS and enzymatically detached from the plate, preferably with an enzyme such as trypsin, TrypLE® or the like according to standard protocols. Afterwards, growth medium is added, the MPCs are centrifuged, and seeded on plates at a density of 3000-7000 cells/cm². In one embodiment of the present invention, the MPCs are seeded on plates coated with an extracellular matrix protein, such as collagen or fibronectin. In an alternative embodiment, the MPCs are seeded on plates that are not coated with an extracellular matrix protein. Those P1 cells are cultivated with change of the growth medium every 2 to 3 days. Then the cells are either split again or used for seeding in a mass culture system as defined herein. When the cells are detached from the plates after centrifugation and resuspension in growth medium, they are usually counted including determination of cell viability. If necessary, cells can also be frozen according to standard protocols, e.g., for storage, prior to mass cultivation. If cells were frozen they are usually cultured as monolayer for one passage prior to mass cultivation.

Accordingly, in one embodiment, the MPCs to be cultured in accordance with the method of the present invention are first passage (P1) cells, which are preferably obtained as described hereinbefore.

Optionally, re-plating of the MPCs is performed to further expand the cells, preferably the cells are re-plated onto coated, in particular collagen coated dishes and cultivated in growth medium. In an alternative preferred embodiment, the MPCs are re-plated onto uncoated dishes. Thus, in another embodiment of the present invention, the MPCs to be cultured in the method of the present invention are second passage (P2) or third passage (P3) MPC, which are preferably obtained as described hereinbefore. In a preferred embodiment, the MPCs are P1 or P2 cells, more preferably P1 MPCs.

In case the MPCs are not directly transferred to the mass culture system, but are frozen and, for example, stored in liquid nitrogen, in particular cryopreserved in the vapour phase of liquid nitrogen, they are usually cultured as monolayer for one passage on culture dishes before being seeded in the mass culture system. Accordingly, in one embodiment of the present invention, the cells obtained from the biopsy are frozen, e.g., for storage, and after thawing cultivated for one passage as monolayer. In an alternative embodiment, the thawed cells are directly seeded into the mass culture system.

### Mass Culture

The term “mass culture” or “mass cultivation” refers to the expansion of cells in order to obtain an amount of cells large enough for a desired downstream application. Of course, the cell number required for certain downstream applications differs but is known to or conceivable by a person skilled in the art.

The method according to the present invention is exemplarily performed as described in Examples 1 and 2 and results, for example, in about 2.8×10⁸ MPCs in total. Furthermore, as shown in Example 3, the method of the present invention results in MPCs that have the specific myogenic marker profile, including the presence Pax7, α-Actinin, Desmin and absence of CD34. This means that sufficient cells with the required marker expression are obtained for cell therapeutic approaches, such as for the preparation of a medicament and/or composition of the present invention as defined below. As a specific example, the targeted cell count for injection into each patient for the treatment of skeletal muscle dysfunctions, such as urinary incontinence, is preferably in the range of 80-150 million cells in total. However, as outlined, infra, the therapeutic amount highly depends on the indication to be treated.

An exemplary cultivation method of the present invention is depicted in Example 1. In a first step of the method of the present invention, the cell culture medium is inoculated with the MPCs as defined hereinbefore and the MPCs are cultivated in a culture medium comprising microcarriers, wherein the microcarriers provide a growth surface for the MPCs. In one embodiment, the method of the present invention comprises pre-equilibration of the microcarriers with the culture medium under the desired culture conditions, i.e. the microcarriers are added to the culture medium prior to inoculation of the culture medium with the cells and kept in the container in which the cultivation of the MPCs is performed. The culture medium comprising the microcarriers as defined below and, in particular, its volume is herein referred to as “starting culture medium” and “starting volume”, respectively.

In one embodiment, the method of the present invention comprises inoculation of the cell culture medium with at least 10⁵, preferably with 10⁵ to 10⁶ cells in 130 mL culture volume. Of course, dependent on the culture volume, the cell number is adapted so that about 750 cells/ml to 8000 cells/ml are inoculated.

In accordance with the method of the present invention, MPCs are seeded in the container comprising the culture medium and microcarriers at a density between 500-1500 cells/cm² growth surface area provided by the microcarriers. The cells are incubated in the container thereby allowing the cells to attach to the microcarriers. In a preferred embodiment, the cells are seeded at a density between 800-1200 cells/cm² growth surface area provided by the microcarriers. In one embodiment, the concentration of the cells that are seeded in accordance with the method of the present invention is 750-8000 cells/ml, preferably about 7700 cells/ml. In one embodiment, at least 10⁵ MPCs, preferably 10⁵ to 10⁶ MPCs, more preferably about 10⁶ MPCs are seeded in a culture medium volume of 130 ml.

In particular, in one embodiment of the invention, the method of obtaining a mass culture of MPCs comprises at least the following steps:
(a) seeding MPCs in a container comprising culture medium which comprises microcarriers and allowing the MPCs to attach to the microcarriers, wherein MPCs are seeded at a density between 500-1500 cells/cm² growth surface area provided by the microcarriers, preferably at a density between 800-1200 cells/cm², preferably wherein the microcarriers provide a growth area of 5,000-10,000 cm²/L, more preferably of 8,500 cm²/L; and
(b) cultivating the MPCs in the container; and
(c) increasing the growth surface area of the culture medium when the cell number has increased about 8-fold to 25-fold; and
(d) further cultivating the MPCs, preferably until a cell density up to 5-7.5×10⁴ cells/cm² and/or 4-6.5×10⁵ cells/ml has been reached, and optionally
(e) harvesting the MPCs.

In one embodiment of the invention, the method of obtaining a mass culture of MPCs comprises at least the following steps:
(a) seeding MPCs in a container comprising culture medium which comprises microcarriers and allowing the MPCs to attach to the microcarriers, wherein MPCs are seeded at a density between 500-1500 cells/cm² growth surface area provided by the microcarriers, preferably at a density between 800-1200 cells/cm², preferably wherein the microcarriers provide a growth area of 5,000-10,000 cm²/L, more preferably of 8,500 cm²/L; and
(b) cultivating the MPCs in the container; and
(c) increasing the growth surface area of the culture medium when the cell density is 1.3×10⁴-1.8×10⁴ cells/cm²; and
(d) further cultivating the MPCs, preferably until a cell density up to 5-7.5×10⁴ cells/cm² and/or 4-6.5×10⁵ cells/ml has been reached, and optionally
(e) harvesting the MPCs.

In one embodiment of the invention, the method of obtaining a mass culture of MPCs comprises at least the following steps:
(a) seeding MPCs in a container comprising culture medium which comprises microcarriers and allowing the MPCs to attach to the microcarriers, wherein MPCs are seeded at a density between 500-1500 cells/cm² growth surface area provided by the microcarriers, preferably at a density between 800-1200 cells/cm², preferably wherein the microcarriers provide a growth area of 5,000-10,000 cm²/L, more preferably 8,500 cm²/L; and
(b) cultivating the MPCs in the container; and
(c) increasing the growth surface area of the culture medium when at least 70% of the microcarriers are occupied, preferably at least 75%, more preferably at least 80%, more preferably at least 85%, more preferably at least 90%, or 95%, most preferably at least 90%; and
(d) further cultivating the MPCs, preferably until a cell density up to 5-7.5×10⁴ cells/cm² and/or 4-6.5×10⁵ cells/ml has been reached, and optionally
(e) harvesting the MPCs.

In one embodiment of the invention, the method of obtaining a mass culture of MPCs comprises at least the following steps:
(a) seeding MPCs in a container comprising culture medium which comprises microcarriers and allowing the MPCs to attach to the microcarriers, wherein MPCs are seeded in an amount of 750-8000 cells/ml, preferably about 7700 cells/ml, preferably wherein the microcarriers provide a growth area of 5,000-10,000 cm²/L, more preferably 8,500 cm²/L; and
(b) cultivating the MPCs in the container; and
(c) increasing the growth surface area of the culture medium when the cell number has increased about 8-fold to 25-fold; and
(d) further cultivating the MPCs, preferably until a cell density up to 5-7.5×10⁴ cells/cm² and/or 4-6.5×10⁵ cells/ml has been reached, and optionally
(e) harvesting the MPCs.

In one embodiment of the invention, the method of obtaining a mass culture of MPCs comprises at least the following steps:
(a) seeding MPCs in a container comprising culture medium which comprises microcarriers and allowing the MPCs to attach to the microcarriers, wherein MPCs are seeded in an amount of 750-8000 cells/ml, preferably about 7700 cells/ml, preferably wherein the microcarriers provide a growth area of 5,000-10,000 cm²/L, more preferably 8,500 cm²/L; and
(b) cultivating the MPCs in the container; and
(c) increasing the growth surface area of the culture medium when at least 70% of the microcarriers are occupied, preferably at least 75%, more preferably at least 80%, more preferably at least 85%, more preferably at least 90%, or 95%, most preferably at least 90%; and
(d) further cultivating the MPCs, preferably until a cell density up to 5-7.5×10⁴ cells/cm² and/or 4-6.5×10⁵ cells/ml has been reached, and optionally
(e) harvesting the MPCs.

In a preferred embodiment, the growth surface area and optionally the volume of the culture medium is increased between two and fourfold, preferably threefold (step (c)). In order to obtain higher cell numbers, the step of increasing the growth surface can be repeated one or more times, i.e. the growth surface can again be increased between two and fourfold when a certain cell density, cell number and/or fold increase of the cells as defined elsewhere herein has been reached. Therefore, in one embodiment, the present invention comprises one or more steps of increasing the growth area of the culture environment, preferably 1 to 10 steps of increasing the growth area, preferably 1 to 8 steps, more preferably 1 to 4 steps, more preferably 1 or 2 steps, most preferably 2 steps of increasing the growth area.

The term “cultivating” refers to conditions for maintaining and growing cells in cell culture. During the cultivation performed in accordance with the method of the present invention, the container and culture medium, respectively, are kept in intermitted motion in order to keep the microcarriers in suspension. The cells attached to the microcarriers grow optimally when the microcarriers are kept in a homogeneous suspension and do not settle or sediment except where needed to facilitate migration of cells over the microcarriers. In particular, keeping the microcarriers in suspension and also in motion avoids substantial cell aggregation that could possibly lead to MPC differentiation or senescence. The force applied to keep the microcarriers in suspension should be such that the microcarriers do not settle or sediment. However, the force should not be too much so to damage the cells or microcarriers. Different possibilities for keeping the microcarriers in suspension/in motion exist and include but are not limited to stirring of the culture medium and rocking the cell culture system, in particular rocking the bioreactor as described in Example 1.

The typical cell culture environment for mammalian cells is known to a person skilled in the art. For example, the pH range for cultivation of mammalian cells is usually between 7.2 and 7.6 and the temperature is usually between 36° C. and 37° C. This was confirmed by the experiments performed in accordance with the present invention, where growth was observed at pH 7.3 and 37° C. Accordingly, in one embodiment, the pH is between 7.2 and 7.6, but preferably the pH is kept at 7.3 to 7.4, most preferably to 7.3 and the temperature set point is between 36° C. and 37° C., preferably the temperature is kept at 37° C. The dissolved oxygen (DO) concentration is usually kept between 20% and 80%, preferably between 30% and 75%. In particular, the DO set point in the method of the present invention is set to 75% and should not drop below 30%.

Experiments performed in accordance with the present invention showed that it is beneficial when the cells are seeded in a smaller volume (starting volume) to increase the concentration of the culturing cells. After some time, during expansion of the cells, more growth area is required for which reason the growth surface is increased by addition of further microcarriers. Accordingly, the method of the present invention comprises the addition of cell culture medium comprising microcarriers to the cell culture to increase the growth surface. The culture medium after the increase and, in particular, its volume is herein referred to as the “expansion culture medium” and “expansion volume”, respectively. In a preferred embodiment, the concentration of the microcarriers in the expansion culture medium is roughly the same as in the starting culture medium, preferably between 1-2 g/L, more preferable about 1.7 g/L. In accordance with the present invention, the volume of the culture medium is increased once the cell number has increased about 8-fold to 25-fold and/or when more than 80% of the microcarriers are occupied, preferably more than 90%. In particular, increasing the volume of the culture medium while maintaining the microcarrier concentration means that more growth area is provided for further expanding the cells. In an alternative embodiment, the growth surface is increased by addition of microcarriers to the container while the culture medium is not increased to the same extent, i.e. the resulting concentration of the microcarriers is higher or lower than before.

A convenient marker to increase the size of the expansion volume is after more than 50% of the microcarriers are occupied, preferably at least 75%, 80% or 85%, most preferably at least 90%.

In a preferred embodiment, the method of the present invention comprises an increase of the volume of the culture medium and/or growth surface area when a cell density of 1.3×10⁴-1.8×10⁴ cells/cm² is reached.

In one embodiment, the method of the present invention comprises a two- to four-fold increase, preferably an approximately three-fold increase of the growth area. For example, the culture volume is increased about three-fold and the microcarrier concentration is roughly maintained in the expansion culture medium, i.e. a culture medium containing microcarriers in about the same concentration as during seeding is added.

In one embodiment of the present invention, the starting volume, i.e. the volume of the culture medium which is inoculated with the MPCs is about 100 to 150 ml, preferably about 130 ml. In a preferred embodiment, the starting volume is increased to 400 ml expansion volume.

In one embodiment, the method of the present invention further comprises a step of refreshing the culture medium after increasing the growth surface, for example, every second day during the further cultivation of the cells in order to supply the MPCs with sufficient nutrients.

The cultivation time can vary and depends on the cell densities as specified, supra. Preferably the MPCs are cultured 5-21 days, more preferably 7-14 days, more preferably 8-10 days. However, longer or shorter cultivation periods are conceivable depending on the number of cells seeded and/or their growth rate and/or the desired final cell number.

In accordance with the method of the present invention, the cultivation is ended once the desired cell number is reached, preferably once the cell number has increased about 100-fold to 1000-fold and/or once the cell density has increased about 10-fold to 200-fold, preferably about 13-fold to 130-fold. In particular, the MPCs are further cultivated until they reach a number suitable for further subsequent therapeutic approaches, i.e. in particular until a cell density up to 5-7.5×10⁴ cells/cm² and/or 4-6.5×10⁵ cells/ml has been reached. In a preferred embodiment, the cells are cultivated until a density of 5.7×10⁵ cells/ml, which has been found to be an optimal density that supports maintenance of MPC characteristics, i.e. for example no fiber-formation. In one embodiment in accordance with the present invention, the MPCs are cultivated until a total cell number of about 1.5×10⁸-2.75×10⁸ has been obtained, preferably about 2.3×10⁸ cells in total. However, if the required cell number cannot be obtained with one expansion step, i.e. increasing the culture volume once, further expansion steps may be performed, thereby further increasing the culture volume and growth area, preferably 3-fold in each expansion step, while the concentration of the microcarriers is preferably maintained the same.

In one embodiment, the MPCs are harvested after the desired cell number and cell density, respectively has been reached. In particular, in one embodiment, the method of the present invention comprises a step of detaching the MPCs from the microcarriers at the end of the cultivation by cleavage of the anchorage proteins via enzymatic or mechanical means (see above) as well as a step of retrieving the MPCs from the bioreactor. In a preferred embodiment, the detachment comprises a complete dissolution of the microcarriers. In case non-dissolvable microcarriers are used, the cells are separated from the microcarriers quickly before the cells start to attach again.

Since MPCs are grown on microcarriers in a suspension culture setting similar to that of non-adherent cells, it is conceivable that volume and/or growth surface of the culture system can be further increased in further expansion steps in order to obtain even larger number of cells.

### Microcarriers

In accordance with the present invention, microcarriers in general refer to support for cultivating anchorage-dependent cells. “Microcarrier” or “carrier particle” is defined as small, beaded material, derived from silica, glass, dextran or similar materials, used for the immobilization of biocatalysts or as a support for the culture of anchorage-dependent animal cell lines (IUPAC Compendium of Chemical Terminology (2nd Edition, 1992, Vol. 64, p. 160). Microcarriers increase the growth surface area in a tissue culture for the attachment and yield of anchorage-dependent cells. The terms “growth surface are”, “growth area” and “surface area” are used interchangeably herein and refer to the area of the surface the microcarriers provide which anchorage-dependent cells attach to for being cultivated. Microcarriers used in accordance with the present invention are carrier materials, preferably spherical in form and suitable for cultivating adherent growing cells, in particular animal cells, in suspension.

Microcarriers may be produced from a wide variety of materials, including plastic, glass, ceramic, silicone, gelatin, dextran, cellulose and others. In addition, microcarriers can be pretreated in various ways including plasma treatment of the plastic surfaces that results in creating a hydrophilic surface, or the carriers can be coated (e.g. with gelatin, fibronectin, laminin, polyomithine, matrigel, or with binding motifs of the RGD binding domain of fibronectin). In a preferred embodiment, the microcarriers as used in accordance with the present invention are made of polygalacturonic acid (PGA) polymer chains cross linked via calcium ions and coated with denatured collagen. Suitable commercially available microcarriers include Cytodex™ 1, Cytodex™ 3, Cytopore™ (Amersham Biosciences), Cultispher® G, Cultispher® S (Perbio), Pronectin®, FACT (Sigma), Biosilon®, Microhex™ (Nunc), ImmobaSil™ (Dunn), and collagen-coated (dissolvable) microcarriers (Corning™).

In one embodiment, the microcarriers as used in accordance with the method of the present invention are collagen-coated microcarriers. The microcarriers can be dissolvable or non-dissolvable microcarriers. In a preferred embodiment, the microcarriers are dissolvable and thus, the microcarriers are preferably collagen-coated dissolvable microcarriers. In case the microcarriers are dissolvable, in one embodiment of the present invention, the dissolution of the microcarriers is performed by enzymatic digestion, preferably by the addition of a harvest solution comprising a peptidase, preferably an endopeptidase that cleave proteins at specific sites, most preferably trypsin, or a corresponding trypsin substitute like TrypLE®, or Accutase®, and pectinase. TrypLE® cleaves peptide bonds at the C-terminal end of lysine and arginine and can be used as a direct replacement for trypsin and is of animal-free origin. Accutase® is a natural enzyme mixture with proteolytic and collagenolytic enzyme activity. This means it mimics the action of trypsin and collagenase at the same time. Accordingly, in a preferred embodiment, the harvest salutation comprises either trypsin and a pectinase, or TrypLE® and a pectinase, or Accutase® and pectinase, most preferably TrypLE® and a pectinase. In one embodiment, the above-mentioned harvest solution further comprises EDTA which is helpful for the complete dissolution of the microcarriers.

In one embodiment, the concentration of the microcarriers as used in accordance with the method of the present invention in the medium is about 0.5 to 3 g/L, preferably about 1-2 g/L, more preferably 1.7 g/L, wherein the bead size is preferably 100 to 400 μm, preferably 200-300 μm fully hydrated, and the surface is preferably 1000-10,000 cm²/gram dry weight, more preferably 3000-8000 cm²/gram dry weight, more preferably 4000-7000 cm²/gram dry weight, and most preferably 5000 cm²/gram dry weight. In general, the concentration of the microcarriers in the culture medium depends on the specific microcarriers used. For example, a growth surface of 5,000-10,000 cm²/L of culture medium should be provided by the microcarriers. In a preferred embodiment, the microcarriers provide a growth surface of about 8500 cm²/L of culture medium. This corresponds to the use of 1.7 g/L microcarriers with a surface of 5,000 cm²/g.

The purpose of the microcarriers is to provide an increased growing surface for the adherent cells. Therefore in another preferred embodiment, the microcarriers provide a growth surface area from 100 to 60,000 cm², more preferably a growth surface area from 500 to 40,000 cm², most preferably a growth surface area from 1,000 to 20,000 cm². It may be clear that as the cells are increasing in number, additional microcarriers may be added to provide enough growth surface area. Depending on the amount of surface area occupied by the adherent cells, microcarriers may be added during the culturing, for example in the expansion steps.

After the cells have been grown until a desired amount of cells is achieved, i.e. the amount of cells as defined hereinbefore, the MPCs are harvested, i.e. recovered from the culture system. In one embodiment of the present invention, the adherent cells, i.e. MPCs are detached from the microcarriers. The detachment may be done with a suitable detachment agent. Suitable detachment agents may be enzymes, thermos-responsive agents and or pH-responsive agents. In one embodiment of the present invention, the MPCs are detached with a digestive enzyme cleaving the cells from the microcarrier, preferably wherein the enzyme is endopeptidase, and more preferably selected from trypsin, TrypLE®, or Accutase®. In case non-dissolvable microcarriers are used, the adherent cells that have been detached from the microcarriers may be removed through a 50-100 μm filter. The detached adherent cells will pass through the filter while the microcarriers are retained in the container. In case of the dissolvable microcarriers, the harvesting solution as referred to above can be use.

In an alternative embodiment of the present invention, the MPCs are harvested without detachment from the microcarriers, i.e. the MPCs retrieved from the culture system comprise the microcarriers. Thus, the cells can be directly prepared for injection without detachment, in particular if the carriers are biocompatible and/or biodegradable, like collagen carrier. Accordingly, in case of biocompatible/biodegradable microcarriers, cells are not detached and injected directly while adherent to microcarriers.

### Culture Medium

As used herein, the terms “growth medium” and “culture medium” are used interchangeably and refer to a solution comprising components and nutrients that support viability and proliferation of the cells to be cultured in accordance with the present invention. Suitable culture media for growing MPCs are known to a person skilled in the art and are for example described in WO 1999/056785 A2, WO 2001/078754 A2, WO 2008/066886 A2, WO 2008/086040 A1, WO 2009/045506 A2, and WO 2019/115790 A1.

Growth medium supplements originating from animal origin, such as fetal bovine serum (FBS), are still widely used in cell culture to promote cell attachment, proliferation and maintenance. However, those reagents should be avoided in clinical use because of safety issues. Xeno- and serum-free reagents are highly desirable for enhancing the safety and quality in cell therapeutic methods. Possible alternatives to FBS are media complemented with human serum, human platelet derivatives, allogenic umbilical cord blood serum or chemically defined media. Therefore, in one embodiment, the culture medium as used in accordance with the method of the present invention is a substantially xeno- and/or serum-free medium, e.g., as the culture medium disclosed in WO 2019/215090 A1 which is herein incorporated by reference. Xeno- and/or serum-free refers to the replacement of serum, such as FBS by hPL.

In particular, in one embodiment, a growth medium comprising human platelet lysate (hPL), preferably pooled human platelet lysate (phPL), which preferably has been filtrated, is used in accordance with the present invention. In one embodiment, the final concentration of phPL in the growth medium as used in accordance with the present invention is at least 5%, preferably about 5-20%, more preferably 7-12%, most preferably about 10% or about 5% (volume percent). A concentration of 5% of this hPL has been shown to minimize aggregation of the microcarriers. In a certain embodiment, the culture medium comprises an anti-coagulation factor, preferably heparin. For this purpose, e.g. Heparin-Na (heparin-sodium) (25 00 IU/5 ml) can be used. The heparin is added to the filtrated phPL thus forming a mixture, before adding said mixture to the nutrient solution of the growth medium to a preferred final concentration of 1-10 IU per ml of growth medium, 2-6 IU/ml, or about 2 IU/ml. As an alternative, other substances preventing clotting (e.g. EDTA) can be used. In case of the use of fibrinogen-depleted phPL, no anti-coagulant has to be added, as no active coagulation factors are present anymore.

Therefore, in a preferred embodiment, the cell culture medium as used in accordance with the present invention comprises fibrinogen-depleted human platelet lysate (hPL), and is devoid of heparin.

The cell culture medium may further comprise the following ingredients:
a nutrient solution, preferably Dulbecco's Modified Eagle Medium (DMEM), more preferably a 1:1 DMEM/F12 nutrient mix (1:1 mix of DMEM and Flam's F-12);
human Epidermal Growth Factor (hEGF), preferably added to the nutrient solution to result in a final concentration of 2-20 ng/ml, more preferably about 10 ng/ml;
human basic Fibroblast Growth Factor, (hbFGF), preferably added to the nutrient solution to result in a final concentration of 0.5-2 ng/ml, more preferably of about 1 ng/ml;
insulin, preferably human insulin, preferably added to the nutrient solution to result in a final concentration of 5-20 μg/ml, more preferably of about 10 μg/ml
dexamethasone, preferably added to the nutrient solution to result in a final concentration of 0.2-0.8 μg/ml, more preferably of about 0.4 μg/ml.

According to a one embodiment, the cell growth medium further comprises a solution containing an antibiotic agent, preferably containing penicillin and streptomycin, preferably at a final concentration of about 1% (Pen/Strep: 10000 units/ml of penicillin and 10000 μg/ml of streptomycin in a 10 mM citrate buffer (for pH stability) at 20° C.). In an alternative embodiment, the growth medium is free of penicillin and streptomycin. In an alternative embodiment, the growth medium comprises an antibiotic agent that is not penicillin and/or streptomycin but another antibiotic agent. Further antibiotics and their usage in cell culture mediums are well known to the person skilled in the art and can be used in accordance with the present invention.

### Bioreactor

In order to maintain optimal growth conditions, i.e. in terms of oxygen, carbon dioxide and nutrient supply, all of the above disclosed embodiments of the method of the present invention can conveniently be performed in a bioreactor system as exemplarily shown in Example 1 and 2. In general, a bioreactor is understood as a container suitable for the cultivation of biological material such as cells. One form of a bioreactor is a stir tank. In one embodiment of the method of the present invention, the container is a stirred-tank reactor. Bioreactors are known to the person skilled in the art as systems, in particular, closed systems for the cultivation of cells wherein growth parameters such as dissolved oxygen concentration, the temperature, and pH can be controlled.

Accordingly, in one embodiment, the container as used in the method of the present invention is a closed bioreactor. Preferably, the closed bioreactor is a bioreactor bag. A bioreactor bag suitable for performing the method of the present invention is disclosed in the international application WO 2011/142667 A1, for example in the Examples section “Expansion in culture bags”; the teaching of which is herein incorporated by reference. Advantageously, in one embodiment of the present invention, the bioreactor bag is expandable, i.e. the culture volume can be expanded, for example, to achieve the increase in growth area as described, supra.

The MPCs are transferred to the container in a sterile manner. Preferably, the MPCs are transferred via a bag with weldable tubing, preferably by first aspirating under sterile conditions a cell suspension comprising the MPCs into a syringe and then, under sterile conditions, injecting the cell suspension into the bag, followed by welding of the weldable tubing to the container of the bioreactor system and thereby transferring the cell suspension into the container of the bioreactor system.

When a gas volume or headspace is present in the container, due to the moving of the container, extra turbulence is created in the culture medium. The extra turbulence may cause cells to die or negatively influences cell growth, especially sensitive cells. Therefore in a preferred embodiment, less than 20% headspace, preferably less than 10%, even more preferably no headspace is present in the container. In the present invention, headspace means the percentage of volume of the container containing gas. 20% headspace means that 80% of the container consists of medium with cells and microcarriers.

Additional nutrients and/or supplements may be added during the culturing depending on the needs of the cells. This may be done by perfusion. Sensors may be added to the bioreactor so that they measure the nutrient level and/or waste level, pH, DO (dissolved oxygen), the amount of cells in the system and/or other parameter. Preferably these sensors operate automatically, more preferably the addition of nutrients and/or supplement is also carried out automatically, most preferably the sensors direct the addition of nutrients and/or supplements.

In order to supply the cells with sufficient nutrients, preferably fresh medium is passed through the container while simultaneous medium is removed so that a constant volume and/or pressure are maintained. By removing medium from the expansion container through a filter with a pore size bigger than cell, but smaller than microcarrier, for example with a pore size of about 100 μm, non-attached cells and cell debris are removed, and the adherent cells are retained in the container.

In one embodiment, the method of the present invention comprise the replacement of the culture medium every second day with fresh medium after the increase of the growth surface, preferably wherein 50% of the culture medium is replaced.

In a specific embodiment, the method of the present invention comprises, for example (but not necessarily) the following steps for cultivating the MPCs:
(i) the container is pre-equilibrated before cultivating the MPCs, wherein the dissolved oxygen concentration (DO) is set to 75%, the temperature is set to 37° C. and the pH is set to 7.3;
(ii) the MPCs are cultivated in the container for the first 24 hours or until the DO drops below about 30% (whichever occurs earlier) without perfusion, pH control, and DO control in order to allow the MPCs to attach to the carrier particles (pH and DO are not controlled but measured);
(iii) 24 hours after start of the cultivation or when the DO drops below about 30% (whichever occurs earlier), DO control, pH control and perfusion is initiated, wherein the DO is set to 75%, the pH is set to 7.3, and perfusion is performed with 3 mL/min, preferably wherein the pressure inside the bag is maintained between 80 and 120 mbarg; and/or
(iv) the container is rocked, preferably with the following set points:
   rocker speed of 90°/s;
   maximum tilt angle of 180°;
   acceleration of 90°/s²;
   deceleration of 90°/s²;
   vertical hold time of 10 s;
   four 1-hour static intervals during a 24 hour period (Horizontal hold time: 3600 s (horizontal pause); Number mix cycles: 1000 (horizontal pause)]

In this embodiment, the static intervals may be stopped if the DO drops below 40% or when a cell density reaches 5.0×10⁵ cells/ml or when at least 70%, preferably 80%, more preferably 90% of the microcarriers are occupied (to a horizontal hold time of 0 s and 0 mix cycles during the horizontal pause). In addition or alternatively, the perfusion may be increased to a maximum of 10 ml/min if the set point for DO and/or pH cannot be attained anymore. In addition or alternatively, the acceleration and deceleration are each adjusted to 210°/s² if inhomogeneous mixing is observed. However, the rocking scheme can be adapted as also described in WO 2011/142667 A1.

### Cell Population and Therapeutic Aspects

The presence of the MPC characteristic markers can be verified by a person skilled in the art. For example, WO 2019/215090 A1 discloses in vitro and in vivo analyses such as the presence of marker proteins by flow cytometry, fiber formation by Giemsa staining and transplantation experiments.

MPCs obtainable by the method of the present invention, i.e. the cell population comprising MPCs and harvested, show the following myogenic marker expression:
the population comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells; and/or
the population comprises at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells; and/or
the population comprises at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells; and/or
the population comprises less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells; and optionally
the population comprises Desmin positive cells, preferably between 5% to 95%, more preferably between 10% to 99%, more preferably between 20% to 95%, more preferably at least 70%, more preferably at least 75%, more preferably at least 90% and most preferably between 70% and 95%.

Accordingly, in one embodiment, the cell population comprises ≥40% α-Actinin positive cells, ≥60% Pax7 positive cells, ≤20% CD34 positive cells, and optionally Desmin positive cells, preferably ≥50% α-Actinin positive cells, ≥60% Pax7 positive cells, ≤15% CD34 positive cells, and optionally Desmin positive cells.

Preferably, the cell population comprises more than 80% α-Actinin positive cells, more than 80% Pax7 positive cells, less than 5% CD34 positive cells, and optionally Desmin positive cells, most preferably more than 10% Desmin positive cells.

In one embodiment, the cell population comprises ≥40% α-Actinin positive cells, ≥60% Pax7 positive cells, ≥50% A2B5 positive cells, ≤20% CD34 positive cells, and optionally Desmin positive cells, and preferably ≥50% α-Actinin positive cells, ≥60% Pax7 positive cells, ≥50% A2B5 positive cells, ≤15% CD34 positive cells, and optionally Desmin positive cells.

Preferably, the cell population comprises more than 80% α-Actinin positive cells, more than 80% Pax7 positive cells, more than 80% A2B5 positive cells, less than 5% CD34 positive cells, and optionally Desmin positive cells, most preferably more than 10% Desmin positive cells.

In general, the cells of population of the present invention also express early myogenic markers so that the cells when administered to a patient result in muscle regeneration, i.e. are capable of inducing muscle formation. In one embodiment of the present invention, the MPCs express MyoD and/or MyHC, preferably next to the other markers mentioned above.

Phrases regarding the percentage expression of markers such as that the population comprises a percentage of positive cells reflects the proportion of cells positive for the marker in the total population. If a cell expresses a marker, i.e. the marker can be detected on protein or mRNA level by any suitable detection method, such as flow cytometry, Western Blot, immunostaining, or qPCR, the cell is regarded as being positive for the marker.

Therefore, in one embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells. In a further embodiment of the present invention, the cell population of the present invention comprises at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells. In a further embodiment of the present invention, the cell population of the present invention comprises at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells. In a further embodiment of the present invention, the cell population of the present invention comprises less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells.

In a further embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells and at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells. In a further embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells and at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells. In a further embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells and less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells. In a further embodiment of the present invention, the cell population of the present invention comprises at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells and at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells. In a further embodiment of the present invention, the cell population of the present invention comprises at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells and less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells. In a further embodiment of the present invention, the cell population of the present invention comprises at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells and less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells. In a further embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells; at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells; and at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells. In a further embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells; at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells; and less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells. In a further embodiment of the present invention, the cell population of the present invention comprises at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells; at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells; and less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells.

In a most preferred embodiment of the present invention, the cell population of the present invention comprises at least 40% α-Actinin positive cells, preferably at least 50%, more preferably at least 60%, more preferably at least 65%, more preferably at least 70%, more preferably at least 75%, more preferably at least 80%, more preferably at least 85%, more preferably at least 90%, more preferably at least 95%, more preferably at least 98%, and most preferably at least 99% α-Actinin positive cells; at least 60% Pax7 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% Pax 7 positive cells; at least 60% A2B5 positive cells, preferably at least 65%, more preferably at least 70%, more preferably at least 75%, more preferably at least 80%, more preferably at least 85%, more preferably at least 90%, more preferably at least 95%, more preferably at least 98%, and most preferably at least 99% A2B5 positive cells; less than 20% CD34 positive cells, preferably less than 15%, more preferably less than 10%, more preferably less than 8%, more preferably less than 7.5%, more preferably less than 5%, more preferably less than 4%, more preferably less than 3%, more preferably less than 2%, more preferably less than 1.5%, more preferably less than 1%, more preferably less than 0.5%, and most preferably less than 0.25% CD34 positive cells.

In a preferred embodiment, the cell population of the present invention is further defined by the marker(s) CD56, Myf5, MyHC and/or MyoD (preferably in addition to the markers as specified above) with the following percentages: In one embodiment, the cell population of the present invention comprises less than 15% CD56 positive cells, preferably less than 10%, more preferably less than 5%, most preferably about 3% CD56 positive cells. In a further embodiment, the cell population comprises at least 50% Myf5 positive cells, preferably more than 60%, more preferably 60-90%, most preferably about 65% Myf5 positive cells. In a further embodiment, the cell population of the present invention comprises less than 30% MyHC positive cells, preferably less than 20%, more preferably less than 15%, most preferably less than 10% MyHC positive cells. In a further embodiment, the cell population of the present invention comprises 10-40% MyoD positive cells, preferably 10-30%, more preferably 15-25%, most preferably about 20% MyoD positive cells. In a further embodiment, the cell population of the present invention comprises less than 15% CD56 positive cells, preferably less than 10%, more preferably less than 5%, most preferably about 3% CD56 positive cells and at least 50% Myf5 positive cells, preferably more than 60%, more preferably 60-90%, most preferably about 65% Myf5 positive cells. In a further embodiment, the cell population of the present invention comprises less than 15% CD56 positive cells, preferably less than 10%, more preferably less than 5%, most preferably about 3% CD56 positive cells and less than 30% MyHC positive cells, preferably less than 20%, more preferably less than 15%, most preferably less than 10% MyHC positive cells. In a further embodiment, the cell population of the present invention comprises less than 15% CD56 positive cells, preferably less than 10%, more preferably less than 5%, most preferably about 3% CD56 positive cells and 10-40% MyoD positive cells, preferably 10-30%, more preferably 15-25%, most preferably about 20% MyoD positive cells. In a further embodiment, the cell population of the present invention comprises less than 15% CD56 positive cells, preferably less than 10%, more preferably less than 5%, most preferably about 3% CD56 positive cells, at least 50% Myf5 positive cells, preferably more than 60%, more preferably 60-90%, most preferably about 65% Myf5 positive cells, and less than 30% MyHC positive cells, preferably less than 20%, more preferably less than 15%, most preferably less than 10% MyHC positive cells. In a further embodiment, the cell population of the present invention comprises less than 15% CD56 positive cells, preferably less than 10%, more preferably less than 5%, most preferably about 3% CD56 positive cells, at least 50% Myf5 positive cells, preferably more than 60%, more preferably 60-90%, most preferably about 65% Myf5 positive cells, and 10-40% MyoD positive cells, preferably 10-30%, more preferably 15-25%, most preferably about 20% MyoD positive cells. In a further embodiment, the cell population of the present invention comprises less than 15% CD56 positive cells, preferably less than 10%, more preferably less than 5%, most preferably about 3% CD56 positive cells, at least 50% Myf5 positive cells, preferably more than 60%, more preferably 60-90%, most preferably about 65% Myf5 positive cells, less than 30% MyHC positive cells, preferably less than 20%, more preferably less than 15%, most preferably less than 10% MyHC positive cells, and 10-40% MyoD positive cells, preferably 10-30%, more preferably 15-25%, most preferably about 20% MyoD positive cells. In a further embodiment, the cell population of the present invention comprises at least 50% Myf5 positive cells, preferably more than 60%, more preferably 60-90%, most preferably about 65% Myf5 positive cells and less than 30% MyHC positive cells, preferably less than 20%, more preferably less than 15%, most preferably less than 10% MyHC positive cells. In a further embodiment, the cell population of the present invention comprises at least 50% Myf5 positive cells, preferably more than 60%, more preferably 60-90%, most preferably about 65% Myf5 positive cells and 10-40% MyoD positive cells, preferably 10-30%, more preferably 15-25%, most preferably about 20% MyoD positive cells. In a further embodiment, the cell population of the present invention comprises at least 50% Myf5 positive cells, preferably more than 60%, more preferably 60-90%, most preferably about 65% Myf5 positive cells, less than 30% MyHC positive cells, preferably less than 20%, more preferably less than 15%, most preferably less than 10% MyHC positive cells, and 10-40% MyoD positive cells, preferably 10-30%, more preferably 15-25%, most preferably about 20% MyoD positive cells. In a further embodiment, the cell population of the present invention comprises less than 30% MyHC positive cells, preferably less than 20%, more preferably less than 15%, most preferably less than 10% MyHC positive cells and 10-40% MyoD positive cells, preferably 10-30%, more preferably 15-25%, most preferably about 20% MyoD positive cells.

The term “about” especially regarding the percentage of positive cells, is defined to include a variation of 10% more or less positive cells.

In addition, cell viability is at least 80%, at least 85%, at least 90%, at least 95%, or at least 98%, preferably at least 80%. Preferably viability of cells of at least 80% (or higher) is remained when cells and the corresponding cell composition comprising collagen, respectively is stored at 2-8° C. for at least 24 hours, preferably for at least 48 hours, and up to 120 hours.

Experiments performed in accordance with the present invention showed that the amount of Desmin positive cells varies between different MPC isolates already before mass cultivation of the cells, i.e. differs in the cell population obtained from the biopsies of different patients and increases during cultivation with every cell passage. Accordingly, the amount of Desmin positive cells is not a crucial criterion for the cells being suitable for downstream clinical applications as long as Desmin positive cells are present in the population.

Therefore, in a further aspect, the present invention relates to a cell population comprising MPCs obtainable by the method of the present invention as disclosed, supra. The population of MPCs according to the present invention can be used in the manufacture of a medicament. In particular, the population of MPCs according to the present invention can be used in the manufacture of a medicament for treating muscle dysfunction, in particular skeletal muscle dysfunction, in a human patient; see infra. Accordingly, in one embodiment, the population as obtained by the method of the present invention comprises the MPCs in a therapeutically effective amount. Therapeutically effective amount means an amount suitable for the treatment of a muscle dysfunction, such as urinary incontinence. The MPCs are administered at the site of the damaged muscle, preferably by injection, in order to regenerate skeletal muscle tissue. In one embodiment of the present invention, a skeletal muscle dysfunction to be treated is a defect of a sphincter muscle. In a preferred embodiment, the sphincter muscle is selected from the non-limiting group of external and internal urethral sphincter, and external and internal anal sphincter. Accordingly, the indication in connection with a sphincter defect to be treated in accordance with the present invention is an indication related to the above-mentioned sphincter muscles and is selected from but not limited to female and male urinary and fecal incontinence, pathologic reflux in a gastroesophageal reflux disorder. Preferably the urinary incontinence is selected from stress incontinence, urge incontinence, overflow incontinence, total incontinence or a mixed form of stress and urge incontinence. Besides targeting sphincter muscles, the cell population obtained by the method of the present invention is used for targeting defects in other skeletal muscles. For example, following injury of those muscles, it is conceivable that regeneration may be supported, facilitated or initiated by administering MPCs at the site of the muscle damage or injury.

In one embodiment, the MPCs to be administered, preferably injected in accordance with the present invention comprise microcarriers. For example, in the above-mentioned embodiment where the MPCs are not detached from the microcarriers, the population comprising MPCs obtainable by the method of the present invention further comprises microcarriers to which the MPCs are attached. In this embodiment, the MPCs are preferably injected together with the microcarriers. Accordingly, the carriers are preferably biocompatible. Furthermore, it is commonly known that the carriers, for example used here as scaffold, should degrade in a timely manner to ensure proper remodeling of the muscle tissue, and thus the carriers should be preferably biodegradable. Biocompatible and biodegradable microcarriers are known to the person skilled in the art. Examples for biocompatible and biodegradable microcarriers are natural polymers (polysaccharides and proteins) and synthetic polymers (poly(α-hydroxy esters), e.g. Poly-epsiloncaprolactone (PCL), poly(glycolic acid) (PGA), poly(lactic acid) (PLA) and poly(lactic-co-glycolic acid) (PLGA); reviewed in Elmowafy, et al., J. Pharm. Investig. 49 (2019), 347-380.

In an alternative embodiment, the MPCs are administered without the microcarriers, i.e. the MPCs are detached from the microcarriers prior to administration.

In general, the amount of cells considered as therapeutically effective is highly dependent on the indication to be treated as well as on the severity, degree or size of the damage to be treated. For example, it is conceivable that less cells are to be injected in case of mild stress urinary incontinence compared to a severe form thereof. In a preferred embodiment, the amount comprises at least 1×10⁷, preferably 6×10⁷ to 3×10⁸, most preferably 1-3×10⁸ MPCs. Even larger cell number can be obtained by performing more than one expansion step, i.e. the volume of culture medium is increased, preferably 3-fold while the concentration of the microcarriers in the medium is preferably maintained. Accordingly, the present invention also relates to a method for obtaining a therapeutically effective amount of MPCs comprising the steps of the method for obtaining a mass culture of MPCs of the present invention described hereinbefore. As outlined above, the amount of MPCs considered as therapeutically effective in general is highly variable and, therefore, not particularly limited. In the specific example of stress urinary incontinence, a targeted cell count for injection into a patient is preferably in the range of 60-200 million cells in total, more preferably about 80-150 million cells. However, as said, those numbers depend on the severity of the defect to be treated. In a preferred embodiment, the viability of the cells is at least 80%.

The present invention further relates to a method of preparing a medicament comprising the steps of the method of obtaining a mass culture of MPCs of the present invention which is described hereinbefore and optionally adding a biomaterial solution, preferably a hydrogel solution, more preferably a collagen solution to the harvested MPCs. A “biomaterial solution” or “biocompatible material” refers to a carrier solution inter alia ensuring that the injected MPCs remain at the injection site. Accordingly, in one embodiment of the present invention, the MPCs are suspended in a biomaterial solution, such as a hydrogel. In general, hydrogels are ECM proteins used in tissue engineering enabling superior engraftment. In a preferred embodiment, the hydrogel is selected but not limited to collagen, alginate, hyaluronic acid, fibrin, poly(N-isopropylacrylamide) (PNIPAAm), poly(ethylene glycol) (PEG), recombinant protein polymers that form Mixing-Induced Two-Component Hydrogels (MITCH), Shear-thinning Hydrogel for Injectable Encapsulation and Long-term Delivery (SHIELD), preferably collagen. Without being bound by theory it is conceivable that in case the MPCs are administered together with, i.e. while being attached to, microcarriers, the microcarriers (as the biomaterial solution) serve as support for the injected MPCs, for example for ensuring that the injected cells remain at the injection site.

The medicament can be used in the treatment of skeletal muscle dysfunction by injection. To deliver 80 million MPCs with at least 80% viability, in a final concentration of 20-25 million cells/ml, the cultured cells (80-100 million) are suspended in one embodiment of the invention in 4 ml of a biomaterial solution, such as a collagen solution as described, infra. The final product is preferably transported in a vial or a syringe in a box at 5° C. (+/−3° C.) controlled by a temperature measuring device. A syringe is defined as container suitable for injecting a medicament into a patient. Alternatively, the MPCs and biomaterial solution are kept separate in the syringe, for example, in a dual chamber syringe, and are only mixed during injection of the medicament.

The present invention further encompasses a composition comprising the above described MPCs and the above-described cell population, respectively, obtained by the method of the present invention, wherein in a preferred embodiment the MPCs are suspended in a collagen solution, preferably at a concentration of 10-30 million cells/ml with at least 80% viability. In a preferred embodiment, the collagen solution contains type I collagen, preferably of porcine, bovine or preferably human origin, and wherein the concentration of collagen in the composition is preferably 1-4 mg/ml, preferably about 2 mg/ml (e.g., 2.1 mg/ml). In one embodiment, the composition is comprised in the pharmaceutical container as defined hereinbefore, preferably in a syringe or a vial.

The MPCs as obtained by the method of the present invention and the corresponding composition of the present invention can be used in various therapeutic applications related in particular to muscle dysfunctions, which include but are not limited to the treatment of stress urinary incontinence as described in WO 2019/215090 A1, the treatment of male stress urinary incontinence after prostatectomy as for example described in WO 2004/096245 A2, and the treatment of anal incontinence as described for example in WO 2008/104883 A1. Thus, the present invention relates to the MPCs and the composition of the present invention, respectively for use as a medicament, preferably in the treatment of a muscle dysfunction, for example a skeletal muscle dysfunction as defined hereinbefore. In a preferred embodiment, the skeletal muscle dysfunction can be for example a dysfunction of the external urethral sphincter muscle or a dysfunction of the external anal sphincter muscle. In a preferred embodiment, the skeletal muscle dysfunction is a defect of the external urethral sphincter muscle and thus, the MPCs and the composition, respectively, is preferably used for the treatment of urinary incontinence, in particular female stress urinary incontinence.

Treatment is usually performed by injecting the composition or the medicament described above into a subject, preferably a mammalian, more preferably a domestic animal, for example pet or livestock as defined before, and most preferably into a human. In one preferred embodiment, treatment is performed by injecting the composition or the medicament described above into the same subject from which the muscle biopsy was taken, and thus autologous cells are preferably used for the treatment. Optionally, the composition further comprises a collagen solution, which is described hereinbefore. After the injection of the composition, the pelvic floor of the human patient can be subjected to neuro-muscular electromagnetic stimulation (NMES) as described in WO 2019/215090 A1. The strength of the induced electric field at maximum output was 120 V/m at the surface of the stimulation coil. At 5 cm above the stimulation coil, the field measured 22 V/m. NMES-treatment following the injection of the cell suspension can support muscle and nerve regeneration by activating muscle-nerve cross-talk and induces the maturation of neuromuscular junctions. Injection of the composition can be performed with injection devices known in the art. In a preferred embodiment, injection is performed with an injection device as described in PCT/EP2023/074044 filed on Sep. 1, 2023, claiming priority of EP 22 193 690.9, which content is herein incorporated by reference. Preferably, 8-12 or 12-18 aliquots of the hMPC-collagen composition are injected into the pelvic floor, not exceeding a total amount of 6 ml of the composition.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

### Example 1: Protocol for Automated MPC Cultivation in a Bioreactor System

MPCs are obtained after explantation of a muscle biopsy. A total of one million P0 or P1 MPCs are collected in a container with weldable tubing, the adherent bag. The methods for explantation of the muscle biopsy and the harvest of the MPCs from the tissue are described in WO 2019/215090 A1 and herein in the section “Muscle precursor cells”, supra. The adherent bag is then welded to a pre-equilibrated SCINUS cell expansion system through sterile welding. MPCs are then grown inside the SCINUS until sufficient cells are obtained (approximately 6 days). The SCINUS system is described in WO 2011/142667 A1.

### SCINUS Preparation

A SCINUS system is prepared using the following set-points, volumes and concentrations:
130 mL MPC culture medium (DMEM:F12 (Gibco™, Thermo Fisher Scientific Inc, USA), 5% hPL (human platelet lysate; PLTGold from MillCreek (Rochester, Minnesota, USA), 1 U/μg Penicillin/Streptomycin (Gibco™, Thermo Fisher Scientific Inc, USA), 10 ng/ml hEGF (Sigma-Aldrich, Saint Louis, USA and Merck KGaA, Darmstadt, Germany, respectively), 0.4 μg/mL dexamethasone (Sigma-Aldrich, Saint Louis, USA and Merck KGaA, Darmstadt, Germany, respectively), 1 ng/mL BFGF (Sigma-Aldrich, Saint Louis, USA and Merck KGaA, Darmstadt, Germany, respectively), 10 μg/mL insulin (Sigma-Aldrich, Saint Louis, USA and Merck KGaA, Darmstadt, Germany, respectively))
1.7 gram/L of Corning Life Sciences collagen-coated dissolvable microcarriers (Corning™ Denatured Collagen Dissolvable Microcarriers)
DO set point 75%
pH set point 7.3
Temperature set point 37° C.

Once the set points are achieved, the pre-equilibrated state is maintained until inoculation of the cells on day 0.

### SCINUS—Seeding (Day 0)

Once 1×10⁶ MPCs (P1) are harvested, the container with the cell suspension is welded to the pre-equilibrated SCINUS bioreactor system. The perfusion and parameter (DO and pH) control were turned off. The following setting during the seeding and attachment phase were used:
Perfusion set point: No control
pH set point: No control
DO set point: No control
Volume set point: 130 mL
Rocker set points:
   Rocker speed: 90°/s
   Max angle: 180°
   Acceleration, 90°/s²
   Deceleration, 90°/s²
   Vertical hold time: 10 s
   Horizontal hold time: 3600 s (horizontal pause)
   Number mix cycles: 1000 (horizontal pause).

The pressure inside the bag is maintained between 80-120 mbarg. If the DO drops below 30% the following settings were enabled:
pH: set point 7.3
DO: set point 75%
Perfusion: set point 3 mL/min

### SCINUS—Start Process Control (Day 1 Onwards)

After the seeding and attachment phase, the MPC culture settings were initiated. In general, this phase is initiated 24 hours after inoculation, or when the DO has dropped below 30%, whichever occurs earlier. The following settings were used during this phase:
Perfusion set point: 3 mL/min
pH set point: 7.3
DO set point: 75%
Volume set point: 130 mL
Rocker set points:
   Rocker speed: 90°/s
   Max angle: 180°
   Acceleration, 90°/s²
   Deceleration, 90°/s²
   Vertical hold time: 10 s
   Horizontal hold time: 3600 s (horizontal pause)
   Number mix cycles: 1000 (horizontal pause).

The Pressure should be maintained between 80-120 mbarg.

Because the growth kinetics and characteristics of MPCs can vary significantly between donors, the following parameters were observed during cultivation.
If the DO drops below 40%, or when the biomass sensor indicates a cell density of ≥5.0×10⁵ cells/mL, or when ≥90% of occupied microcarriers is observed, the horizontal pause is stopped by adjusting the following settings:
   Horizontal hold time: 0 s (no horizontal pause)
   Number mix cycles: 0 (no horizontal pause)
If the set point for DO and pH cannot be attained anymore, the perfusion setting is increased up to a maximum of 10 mL/min.
If inhomogeneous mixing is observed, the following rocker settings are adjusted to maintain homogenous mixing:
   Acceleration: 210°/s²
   Deceleration: 210°/s²

### Volume Expansion (Density-Based)

Once the cells reached a density of 1.3×10⁴-1.8×10⁴ cells/cm² (i.e. 1.1×10⁵-1.5×10⁵ cells/ml), the volume of the adherent bag was increased, while the microcarrier concentration was maintained at 1.7 gram/L. The volume was increased roughly 3-fold, from 130 mL to 400 mL. A suspension of microcarriers in MPC culture medium (1.7 gram/L) was welded to the medium inlet of the SCINUS system and added to the bag either through gravity or pumping.

For increasing the microcarrier volume from 130 mL to 400 mL, the following was performed: the volume set point was adjusted to 400 mL and 270 mL microcarriers were added to the system via the inlet. The pressure was maintained between 80-120 mbarg and the rocker, perfusion, pH, DO set points were not changed.

### Medium Refreshment (Timing-Based)

Medium refreshment is required in the days following the volume expansion step. Every second day, 50% of the cell culture medium was refreshed by transferring 200 mL medium from the adherent bag to a waste bag and adding 200 mL fresh MPC culture medium to the bag via the addition inlet. The pressure was maintained between 80-120 mbarg and the rocker, perfusion, pH, DO set points were not changed.

### Harvest (Density-Based)

Once sufficient cells were cultured inside the adherent bag, the cells were harvested from the dissolvable microcarriers through complete dissolution using a harvest solution. To maintain MPC cell characteristics, the cells were cultured up to a maximum density of 6.7×10⁴ cells/cm² (5.7×10⁵ cells/mL, 2.3×10⁸ cells total).

The cells were washed with an equal volume of PBS once and 200 mL PBS were transferred from the adherent bag to a waste bag. Then 200 mL harvest solution (74% PBS, TryplE 2.5X, pectinase 49 U/mL, EDTA 5M) was added to the adherent bag via the addition inlet. The cells were incubated 15-20 minutes at 37° C., thereby the system was rocked every 5 minute with the following rocking settings:
Rocker set points:
   Rocker speed: 90°/s
   Max angle: 180°
   Acceleration, 90°/s²
   Deceleration, 90°/s²
   Vertical hold time: 10 s
   Horizontal hold time: 300 s
   Number mix cycles: 1.

The cells were retrieved from the bag into a bottle/sample bag.

### Example 2: Cultivation of MPCs in a Bioreactor System

MPCs were cultured according to the protocol detailed in Example 1. Passage 3 cells were used. The MPC culture medium was changed in that it was supplemented with 10% hPL (Paracelsus). The remaining components of the culture medium were used as indicated in Example 1. The cultivation method included a second expansion step after day 10. The volume of the culture medium including microcarriers has been increased from 400 ml to 800 ml, resulting in a total surface area of 6800 cm² as indicated in Table 1.

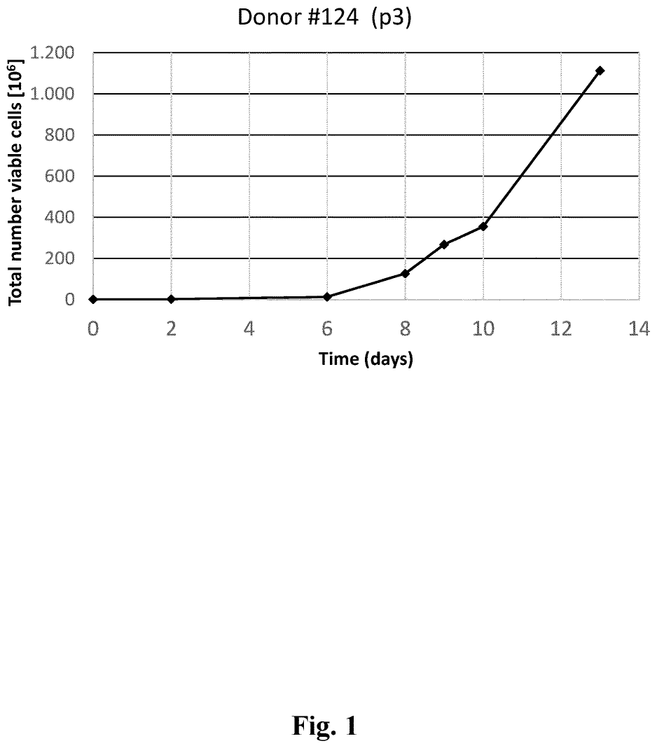

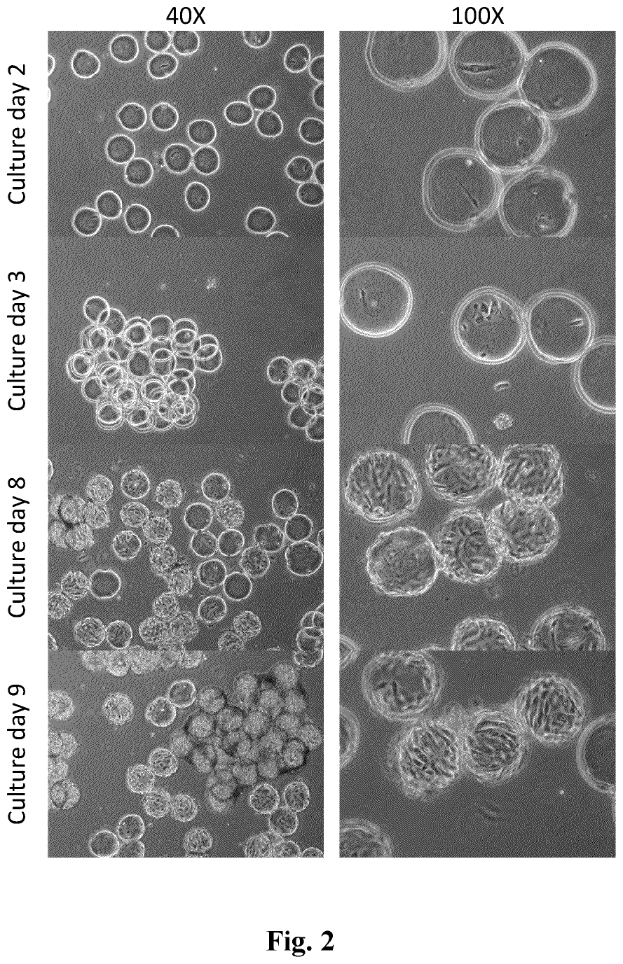

The cells were counted and visualized at least every 2-3 days (Table 1, FIGS. 1 and 2). Therefore, small volume, homogeneous samples were taken from the bioreactor bag. 1 mL was used for visual inspection using light microscopy and images were taken at 40× and 100× magnification. For cell counts, the remaining volume was harvested by dissolving the microcarriers with harvest solution (PBS, TrypLE, EDTA and pectinase). Single cell suspensions were then counted using an NC-250 NucleoCounter. Total cell numbers were adjusted to account for the loss of biomass due to sampling. Expected total cell number is therefore given in the last row of Table 1.

TABLE 1
Cell numbers during cultivation in the above described SCINUS system. Cells were counted at least every 2-
3 days. Total cells numbers were adjusted to account for the loss of biomass due to sampling. Expected total
cell number is therefore given in the last row of the Table.

| | Culture day | | | | | | | |
|---|---|---|---|---|---|---|---|---|
| | 0 | 2 | 3 | 6 | 8 | 9 | 10 | 13 |
| Microcarrier | 1.7 | 1.7 | 1.7 | 1.7 | 1.7 | 1.7 | 1.7 | 1.7 |
| concentration (g/L) | | | | | | | | |
| Total surface area | 1105 | 1105 | 1105 | 1105 | 3400 | 3400 | 3400 | 6800 |
| (cm²) | | | | | | | | |
| Cell count 1 (total) | 7.80E+05 | 2.55E+04 | 9.43E+02 | 8.74E+04 | 2.59E+05 | 4.68E+05 | 6.11E+05 | 9.67E+05 |
| Cell count 1 (total) | 8.14E+05 | 8.02E+03 | 4.72E+03 | 8.96E+04 | 2.46E+05 | 4.70E+05 | 6.26E+05 | 9.39E+05 |
| Viability (average) | 0.97 | 0.935 | 0.85 | 0.974 | 0.991 | 0.986 | 0.975 | 0.978 |
| Average live cells | 7.73E+05 | 1.57E+04 | 2.41E+03 | 8.62E+04 | 2.50E+05 | 4.62E+05 | 6.03E+05 | 9.32E+05 |
| Average adjusted | 7.73E+05 | 1.57E+04 | 2.69E+03 | 1.02E+05 | 3.16E+05 | 6.70E+05 | 8.87E+05 | 1.39E+06 |
| for sampling | | | | | | | | |
| volume (ml) | 130 | 130 | 130 | 130 | 400 | 400 | 400 | 800 |
| Total adjusted for | 1.00E+06 | 2.04E+06 | 3.50E+05 | 1.33E+07 | 1.26E+08 | 2.68E+08 | 3.55E+08 | 1.11E+09 |
| sampling | | | | | | | | |

### Example 3: Characteristics of the MPCs Obtained in the Mass Culture System

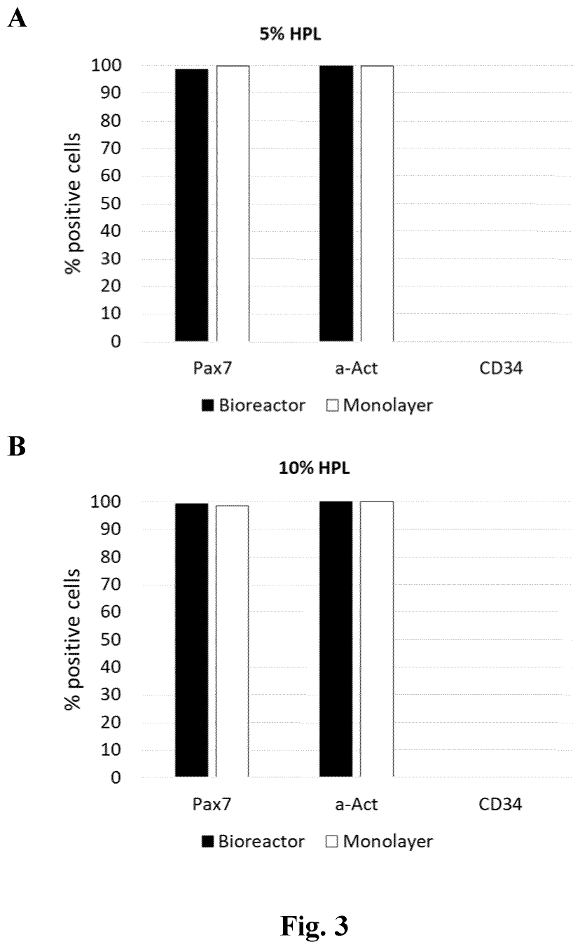

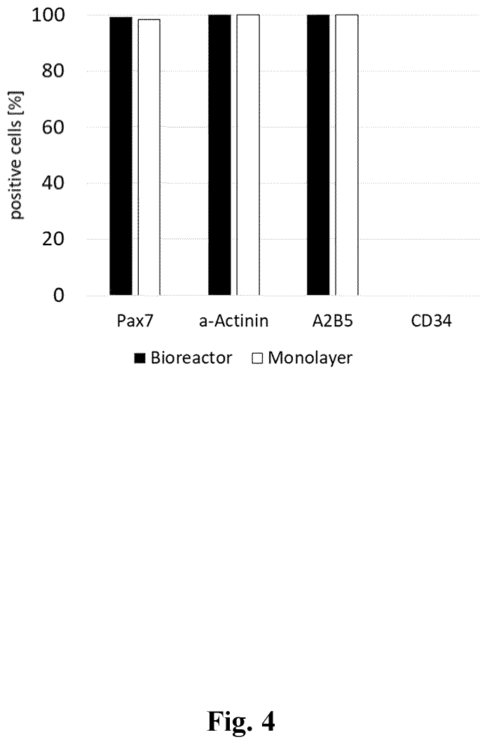

The MPCs cultivated and harvested from the bioreactor are analyzed to confirm that they show myogenic characteristics. Especially for clinical purposes, the MPCs should express ≥50% α-Actinin, ≥60% Pax7, Desmin, and ≤15% CD34. The presence and level of said MPC markers are analyzed by flow cytometry and compared to MPCs cultured as monolayer (FIGS. 3 and 4). In each of the different cultivation methods, the MPC culture medium as described in Example 1 was used which was changed in that it contained 5% PLT Gold HPL (FIG. 3A) and 10% PLT Gold HPL (FIG. 3B and FIG. 4), respectively.

In brief, frozen MPCs were thawed and cultured as monolayer for one passage (P1). Then the cells were seeded in the bioreactor, or T75 monolayer flask. For the bioreactor cultivation, the cells were seeded (1×10⁶ MPCs) and cultured as described in Example 2, i.e. including a second expansion step so that about 160-200×10⁶ cells were obtained.

In principle, the cultivation in monolayer flasks was performed as previously described e.g., in WO 2019/215090 A1.

Harvested cells were analyzed for marker expression by flow cytometry. MPCs were fixed with 2% PFA (Alfa Aesar) in PBS for 10 min at RT) and permeabilized (with 0.5% Titron-X-100 (VWR) for 10 min at RT). Unspecific binding sites were then blocked (with 5% FBS (Sigma) in 0.5% Titron-X-100 in PBS for 20-60 min at 2-8° C.). Surface and intracellular staining with directly and unlabeled antibodies/isotype controls for 30 min at 2-8° C.:
APC-CD34 (TFS)/APC-isoCD34=APC-msIgG1 (TFS)
anti-Pax7 (Sigma)/msIgG2a (TFS)
FITC-anti-alpha-Actinin (Miltenyi)/FITC-REA (Miltenyi)
anti-A2B5 (Sigma)
PE-CD56/PE-msIgG1 (Beckman Coulter)
PE-CD105/PE-msIgG1 (Beckman Coulter)
anti-desmin (Sigma)/msIgG1 (Santa Cruz Biotechnology)
human/mouse Myf-5 AlexaFlour488 (R&D Systems)/msIgG2a (TFS)
MyHC anti-human/mouse/rat-APC/REA Control(S), human IgG1-APC (Miltenyi Biotec)
anti-MyoD (BD Biosciences)/msIgG1 (Santa Cruz Biotechnology)

Antibodies were diluted in autoMACS Running Buffer (Miltenyi Biotec). MPCs were stained with FITC-labelled 2^{nd} antibody (BD) for Pax7 and the corresponding isotype controls for 30 min at 2-8° C. Data is acquired with a MACSQuant by Miltenyi using the manufacturer's protocols with MACSQuant Running Buffer, MACSQuant Washing solution, MACSQuant Storage Solution, MACSQuant Calibration Beads and the manufacturer's software (MACS Quantify Software). Data is analyzed using the Flow Jo software.

As can be seen in FIGS. 3 and 4, both of the cultivation methods, i.e. the bioreactor and monolayer flasks resulted in MPCs that express myogenic markers with about 99% of the cells being Pax7, α-Actinin and A2B5 positive and CD34 expression being negative. In addition, the different cultivation setups resulted in a similar population of MPCs for which reason it can be concluded that mass cultivation in a bioreactor is suitable for producing large numbers of MPCs that are suitable for clinical downstream applications.

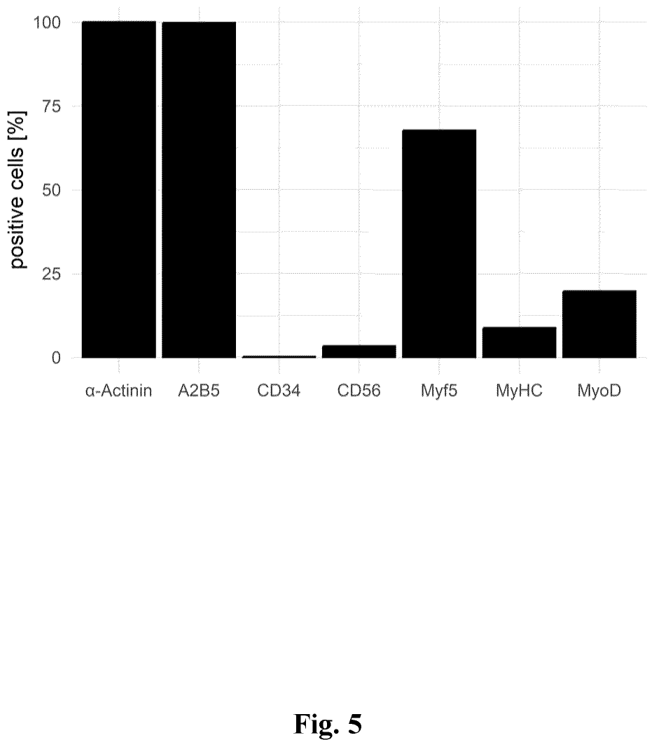

Moreover, as can be seen in FIG. 5, beside displaying the typical myogenic markers α-Actinin and A2B5 (99.9% and 99.7% respectively) and CD34 being negative (0.1%), cells cultivated in the bioreactor are also positive for expression of Myf5, myHC and MyoD (67.6%, 8.7% and 19.6% respectively), and show very low expression of CD56 (3.3%). Desmin expression varied between the different bioreactor runs.

1. A method of obtaining a mass culture of skeletal muscle derived muscle precursor cells (MPCs) comprising at least the following steps:
(a) cultivating MPCs in a container comprising culture medium which comprises microcarriers under conditions allowing the MPCs to attach to the microcarriers, wherein MPCs are seeded at a density between 500-1500 cells/cm² growth surface area provided by the microcarriers, preferably at a density between 800-1200 cells/cm²; and
(b) increasing the growth surface area of the culture medium when the cell number has increased about 8-fold to 25-fold; and
(c) further cultivating the MPCs, preferably until a cell density up to 5-7.5×10⁴ cells/cm² and/or 4-6.5×10⁵ cells/ml has been reached, and optionally
(d) harvesting the MPCs.

2. The method according to claim 1, wherein the growth surface is increased between two and fourfold, preferably about threefold, preferably wherein the volume of the culture medium is increased to the same extent as the growth area.

3. The method according to claim 1 or 2, wherein steps (b) and (c) are repeated one or more time, preferably once or twice, most preferably twice.

4. The method according to any one of claims 1 to 3, wherein the starting volume of the culture medium is about 100 to 150 ml and the volume of the culture medium is increased:
(i) in a first step (b) to 400 ml, and optionally
(ii) in a second step (b) to 800-1000 ml, preferably 1000 ml,
preferably wherein the further cultivating in step (c) is performed until a total cell number of about 1.5-2.75×10⁸ has been obtained.

5. The method according to any one of claims 1 to 4, wherein the container is a closed bioreactor, preferably a bioreactor bag.

6. The method according to any one of claims 1 to 5, wherein the container is an expandable container, preferably an expandable bioreactor bag.

7. The method according to any one of claims 1 to 6, wherein the microcarriers are coated microcarriers, more preferably wherein the microcarriers are collagen-coated microcarrier, and/or wherein the microcarrier are dissolvable.

8. The method according to any one of claims 1 to 7, wherein the culture medium comprises human platelet lysate (hPL).

9. The method according to any one of claims 1 to 8, wherein at the end of cultivation, the MPCs are separated from the microcarriers and harvested, preferably, wherein the separation comprises a complete dissolution of the microcarriers, preferably wherein the dissolution of the microcarriers is performed by enzymatic digestion, preferably by the addition of an endopeptidase that cleave proteins at specific sites, most preferably by trypsin, or a corresponding trypsin substitute, and pectinase.

10. A cell population comprising MPCs obtainable by the method of any one of claims 1 to 9, preferably wherein the cell population comprises ≥40% α-Actinin positive cells, ≥60% Pax7 positive cells, and/or ≤20% CD34 positive cells, and optionally Desmin positive cells, preferably wherein the population comprises ≥50% α-Actinin positive cells, ≥60% Pax7 positive cells, and/or ≤15% CD34 positive cells, and optionally Desmin positive cells, most preferably, wherein the cell population comprises ≥80% α-Actinin positive cells, ≥80% Pax7 positive cells, and/or ≤5% CD34 positive cells, and optionally Desmin positive cells, preferably ≥10% Desmin positive cells.

11. The population of claim 10, wherein the population comprises ≥60% A2B5 positive cells, preferably ≥80% A2B5 positive cells.

12. The population of claim 10 or 11, wherein the population comprises ≤15% CD56 positive cells, preferably ≤10% CD56 positive cells, more preferably ≤5% CD56 positive cells.

13. The population of any one of claims 10 to 12, wherein the population comprises ≥50% Myf5 positive cells, preferably ≥60% Myf5 positive cells, preferably ≥60-90% Myf5 positive cells.

14. The population of any one of claims 10 to 13, wherein the population comprises ≤30% MyHC positive cells, preferably ≤20% MyHC positive cells, preferably ≤15% MyHC positive cells.

15. The population of any one of claims 10 to 14, wherein the population comprises 10-40% MyoD positive cells, preferably 10-30% MyoD positive cells, preferably 5-25% MyoD positive cells.

16. The population according to claim 10, which comprises the MPCs in a therapeutically effective amount, preferably wherein the population comprises at least 1×10⁷ MPCs, preferably 6×10⁷ to 3×10⁸ MPCs, most preferably 1-3×10⁸ MPCs.

17. A method of preparing a medicament comprising the steps of the method of any one of claims 1 to 9, and optionally adding a biomaterial solution, preferably a hydrogel solution, more preferably a collagen solution to the harvested MPCs.

18. The method according to claim 12, which further comprises a step of filling the MPCs into a pharmaceutical container, preferably wherein the container is a syringe or vial.

19. A composition comprising the MPCs obtainable by the method of any one of claims 1 to 9, or the population of MPCs of any one of claims 10 to 16.

20. The composition of claim 19, which further comprises a biomaterial solution, preferably a collagen solution, more preferably at a final concentration of 1-4 mg/mL, more preferably of 2 mg/mL.

21. The composition of claim 19 for use as a medicament, preferably for use in the method of treatment of a skeletal muscle dysfunction, preferably wherein the skeletal muscle dysfunction is a defect of a sphincter muscle, preferably the external urethral sphincter muscle, optionally wherein the composition further comprises a biomaterial solution, preferably a hydrogel solution, more preferably a collagen solution.

22. The composition for use according to claim 21, wherein the method comprises obtaining MPCs from a patient, preparing a muss culture of the MPCs according to the method of any one of claims 1 to 9, preparing a composition according to any one of claims 19 to 21, and administering the composition to the patient, preferably by injection of the muscle to be treated.

23. The composition for use according to claim 21 or 22, wherein the method comprises the following steps:
a) cutting tissue obtained from a muscle biopsy of a patient into small pieces, preferably by using a scissor, preferably wherein the tissue is obtained from a skeletal muscle of said patient, more preferably taken from a tissue selected from the group consisting of: musculus soleus, rectus abdominis, quadriceps femoris, vastus lateralis, and vastus intermedius, preferably from musculus soleus tissue;
b) digesting the tissue biopsy, preferably by a mixture comprising one or more enzymes to disaggregate the tissue, preferably collagenase and dispase;
c) preparing a cell suspension and seeding the cell suspension on coated dishes, preferably on dishes coated with an extracellular matrix protein, preferably with collagen;
d) incubating the cells under appropriate culture conditions allowing fast adhering cells to attach to the dish, preferably at about 36-38° C. for about 20 to 28 h;
e) re-plating the supernatant containing non-adhered cells, mostly MPCs onto dishes coated with an extracellular matrix protein, preferably with collagen, thereby yielding a population comprising MPCs;
f) growing the cells until they reach a number appropriate to be used as inoculum in the method of any one of claims 1 to 9;
g) subjecting the population of MPCs to the method of any one of claims 1 to 9 for obtaining a mass culture of MPCs;
h) preparing a composition by mixing the MPCs with a biomaterial solution, preferably a collagen solution; and
i) administering the composition to the patient, preferably by injection into the muscle to be treated.

24. The composition for use according to any one of claims 21 to 13, wherein the final concentration of collagen in the composition is 1-4 mg/mL, preferably about 2 mg/mL.

25. The composition for use according to any one of claims 21 to 24 wherein the composition comprises 10-30 million cells/ml with at least 80% viability.

26. The composition for use according to any one of claims 21 to 25, wherein the skeletal muscle dysfunction is a defect of a sphincter muscle, preferably the external urethral sphincter muscle, preferably wherein the skeletal muscle dysfunction is urinary incontinence, preferably female urinary incontinence.

27. The composition for use according to any one of claims 21 to 26, wherein the method further comprises subjecting the patient to neuro-muscular electromagnetic stimulation (NMES), preferably wherein the strength of the induced electric field at maximum output is 120 V/m at the surface of the stimulation coil.

28. Use of the composition of claim 19 or 20 for the manufacture of a medicament for a method of treating a skeletal muscle dysfunction, preferably wherein the method is the method as defined in any one of claims 21 to 27.

29. A method of treating a skeletal muscle dysfunction in a patient, wherein the method is the method as defined in any one of claims 21 to 27.
